(19) United States
(12) Patent Application Publication
(10) Pub. No.: US 2024/0047024 A1
(43) Pub. Date: Feb. 8, 2024

(54) METHODS, SYSTEMS, APPARATUSES, AND DEVICES FOR FACILITATING MONITORING AND MANAGING DISEASES OF USERS

(71) Applicants: Jyotsna Mehta, Lexington, MA (US); Shail Dhananjay Mehta, Lexington, MA (US)

(72) Inventors: Jyotsna Mehta, Lexington, MA (US); Shail Dhananjay Mehta, Lexington, MA (US)

(21) Appl. No.: 17/879,849

(22) Filed: Aug. 3, 2022

(51) Int. Cl. G16H 10/60; G16H 40/67

(52) U.S. Cl. CPC G16H 10/60; G16H 40/67; G16H 40/20

(57) ABSTRACT

Disclosed herein is a method for facilitating monitoring and managing diseases of users, in accordance with some embodiments. Accordingly, the method may include steps of receiving disease data associated with a disease of a user from a device, receiving user data associated with the user from the device analyzing the disease data and the user data, generating a report of the user based on the analyzing of the disease data and the user data, processing the report using an executable rule corresponding to the disease, determining a status of the user based on the processing of the report, generating a message for the user and a healthcare provider of the user based on the status, transmitting the message to a user device associated with the user and a healthcare provider device associated with the healthcare provider, and storing the message.

### FIELD OF THE INVENTION

Generally, the present disclosure relates to the field of data processing. More specifically, the present disclosure relates to methods, systems, apparatuses, and devices for facilitating monitoring and managing diseases of users.

### BACKGROUND OF THE INVENTION

In the current healthcare scenario, respiratory health is of paramount importance, especially because respiratory diseases such as asthma are one of the leading causes of death and disability worldwide. According to the World Health Organization (WHO) report on “The Global Impact of Respiratory Disease” published in 2017, an estimated 334 million individuals suffer from asthma. According to the Centers for Disease Control and Prevention (CDC), during the 2019-2020 coronavirus pandemic, individuals suffering from moderate to severe asthma were at a greater risk of falling ill with acute respiratory disease. The global asthma treatment market size is estimated to be around $18.08 billion in 2019 and is projected to reach 26 billion by 2027. The Global Burden of Diseases Study (GBD) 2017 reports indicate 3.2 million deaths due to chronic obstructive pulmonary disease (COPD) and 495,000 deaths due to asthma. COPD was the seventh leading cause of years of life lost (YLLs). Overall, prevalent cases of CRDs were 545 million: about 50% for COPD and 50% for asthma. Incident cases of CRDs were 62 million, mostly due to asthma (69%) and COPD (29%). COPD accounted for 81.6 million disability-adjusted life years, asthma for 22.8 million.

Treatments for lung and breathing disorders depend on the severity and sometimes root cause of the disease: Asthma: The most common treatment for asthma is rescue and controller inhalers, but other treatments and medications can be used. Doctors also recommend patients identify and reduce asthma triggers. Common triggers include allergies, viruses, exercise, cold weather and fumes. Patients are also often taught skills to monitor and manage their asthma. Chronic Cough: Some of the tests that may be used to diagnose the cause of a chronic cough may include a chest X-ray and other radiology tests, breathing tests, pH monitoring, swallow tests and upper GI endoscopy if reflux is associated with the cough. The treatment of chronic cough is usually directed at its cause. However, there is no help to determine best options for treatment. Chronic Obstructive Pulmonary Disease (COPD): The most common cause of COPD is smoking, although breathing in pollutants, dust or chemicals can also be the cause. For smokers, smoking cessation can help prevent the disease or keep it from getting worse. COPD can also be treated with inhalers, medications, oxygen therapy and pulmonary rehab. In severe cases, surgery may be an option. Pulmonary fibrosis is a lung disease that occurs when lung tissue becomes damaged and scarred. This thickened, stiff tissue makes it more difficult for lungs to work properly. The scarring associated with pulmonary fibrosis can be caused by a multitude of factors. But in most cases, doctors can't pinpoint what's causing the problem. When a cause can't be found, the condition is termed idiopathic pulmonary fibrosis.

Therefore, there is a need for improved methods, systems, apparatuses, and devices for facilitating monitoring and managing diseases of users that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is a method for facilitating monitoring and managing diseases of users, in accordance with some embodiments. Accordingly, the method may include a step of receiving, using a communication device, at least one disease data associated with at least one disease of at least one user from at least one device. Further, the at least one disease data may include at least one of an action plan and a prescription for managing the at least one disease. Further, the method may include a step of receiving, using the communication device, at least one user data associated with the at least one user from the at least one device. Further, the at least one user data may include at least one health record of the at least one user. Further, the method may include a step of analyzing, using a processing device, the at least one disease data and the at least one user data. Further, the method may include a step of generating, using the processing device, a report of the at least one user based on the analyzing of the at least one disease data and the at least one user data. Further, the method may include a step of processing, using the processing device, the report using at least one executable rule corresponding to the at least one disease. Further, the method may include a step of determining, using the processing device, a status of the at least one user based on the processing of the report. Further, the method may include a step of generating, using the processing device, a message for at least one of the at least one user and at least one healthcare provider of the at least one user based on the status. Further, the message may include a guidance for the managing of the at least one disease. Further, the method may include a step of transmitting, using the communication device, the message to at least one of at least one user device associated with the at least one user and at least one healthcare provider device associated with the at least one healthcare provider. Further, the method may include a step of storing, using a storage device, the message for at least one of the at least one user and the at least one healthcare provider.

Further disclosed herein is a method for facilitating monitoring and managing diseases of users, in accordance with some embodiments. Accordingly, the method may include a step of receiving, using a communication device, at least one disease data associated with at least one disease of at least one user from at least one device. Further, the at least one disease data may include at least one of an action plan and a prescription for managing the at least one disease. Further, the method may include a step of receiving, using the communication device, at least one user data associated with the at least one user from the at least one device. Further, the at least one user data may include at least one health record of the at least one user. Further, the at least one device further may include at least one respiratory device. Further, the at least one respiratory device may be configured for generating at least one respiratory data of the at least one user based on detecting at least one respiration characteristic of a respiration of the at least one user. Further, the at least one user data further may include the at least one respiratory data. Further, the method may include a step of analyzing, using a processing device, the at least one disease data and the at least one user data. Further, the method may include a step of generating, using the processing device, a report of the at least one user based on the analyzing of the at least one disease data and the at least one user data. Further, the method may include a step of processing, using the processing device, the report using at least one executable rule corresponding to the at least one disease. Further, the method may include a step of determining, using the processing device, a status of the at least one user based on the processing of the report. Further, the method may include a step of generating, using the processing device, a message for at least one of the at least one user and at least one healthcare provider of the at least one user based on the status. Further, the message may include a guidance for the managing of the at least one disease. Further, the method may include a step of transmitting, using the communication device, the message to at least one of at least one user device associated with the at least one user and at least one healthcare provider device associated with the at least one healthcare provider. Further, the method may include a step of storing, using a storage device, the message for at least one of the at least one user and the at least one healthcare provider.

Further disclosed herein is a system for facilitating monitoring and managing diseases of users, in accordance with some embodiments. Accordingly, the system may include a communication device, a processing device, and a storage device. Further, the communication device may be configured for receiving at least one disease data associated with at least one disease of at least one user from at least one device. Further, the at least one disease data may include at least one of an action plan and a prescription for managing the at least one disease. Further, the communication device may be configured for receiving at least one user data associated with the at least one user from the at least one device. Further, the at least one user data may include at least one health record of the at least one user. Further, the communication device may be configured for transmitting a message to at least one of at least one user device associated with the at least one user and at least one healthcare provider device associated with at least one healthcare provider. Further, the processing device may be communicatively coupled with the communication device. Further, the processing device may be configured for analyzing the at least one disease data and the at least one user data. Further, the processing device may be configured for generating a report of the at least one user based on the analyzing of the at least one disease data and the at least one user data. Further, the processing device may be configured for processing the report using at least one executable rule corresponding to the at least one disease. Further, the processing device may be configured for determining a status of the at least one user based on the processing of the report. Further, the processing device may be configured for generating the message for at least one of the at least one user and the at least one healthcare provider of the at least one user based on the status. Further, the message may include a guidance for the managing of the at least one disease. Further, the storage device may be communicatively coupled with the processing device. Further, the storage device may be configured for storing the message for at least one of the at least one user and the at least one healthcare provider.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a block diagram of a system for facilitating monitoring and managing diseases of users, in accordance with some embodiments.

FIG. 3 is a block diagram of the system, in accordance with some embodiments.

FIG. 4 is a block diagram of the system, in accordance with some embodiments.

FIG. 5 is a block diagram of the system, in accordance with some embodiments.

FIG. 6 is a block diagram of the system, in accordance with some embodiments.

FIG. 7 is a block diagram of the system, in accordance with some embodiments.

FIG. 8 is a flowchart of a method for facilitating monitoring and managing diseases of users, in accordance with some embodiments.

FIG. 9 is a continuation flowchart of the method for facilitating the monitoring and managing of the diseases of the users, in accordance with some embodiments.

FIG. 10 is a flowchart of a method for retrieving data for facilitating the monitoring and managing of the diseases of the users, in accordance with some embodiments.

FIG. 11 is a flowchart of a method for determining the status based on remarks of healthcare providers for facilitating the monitoring and managing of the diseases of the users, in accordance with some embodiments.

FIG. 12 is a flowchart of a method for determining the status based on interactions between the users and healthcare providers for facilitating the monitoring and managing of the diseases of the users, in accordance with some embodiments.

FIG. 13 is a flowchart of a method for generating executable rules for facilitating the monitoring and managing of the diseases of the users, in accordance with some embodiments.

FIG. 14 is a flowchart of a method for facilitating monitoring and managing diseases of users, in accordance with some embodiments.

FIG. 15 is a continuation flowchart of the method for facilitating the monitoring and managing of the diseases of the users, in accordance with some embodiments.

FIG. 16 is a flowchart of a method for determining the status based on remarks of healthcare providers for facilitating the monitoring and managing of the diseases of the users, in accordance with some embodiments.

FIG. 17 is a flowchart of a method for facilitating monitoring and managing respiratory diseases of users, in accordance with some embodiments.

FIG. 18 illustrates an action plan for facilitating the monitoring and managing of the respiratory diseases of the users, in accordance with some embodiments.

FIG. 19 illustrates an architecture of a Keva Health Advisor application for facilitating the monitoring and managing of the respiratory diseases of the users, in accordance with some embodiments.

FIG. 20 illustrates a cloud-based system for facilitating monitoring and managing respiratory diseases of users, in accordance with some embodiments.

FIG. 21 illustrates the cloud-based system for facilitating the monitoring and managing of the respiratory diseases of the users, in accordance with some embodiments.

FIG. 22 illustrates a flow diagram of a method for facilitating monitoring and managing respiratory diseases of users, in accordance with some embodiments.

FIG. 23 illustrates a process for generating rules for the engine for facilitating the monitoring and managing of the respiratory diseases of the users, in accordance with some embodiments.

FIG. 24 is a block diagram of a computing device for implementing the methods disclosed herein, in accordance with some embodiments.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of methods, systems, apparatuses, and devices for facilitating monitoring and managing diseases of users, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor, and at least one actuator. Examples of the one or more client devices and/or the server computer may include, a desktop computer, a laptop computer, a tablet computer, a personal digital assistant, a portable electronic device, a wearable computer, a smartphone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server, etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface, etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, a public database, a private database, and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled, and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal, or an artificially intelligent being in any state of existence, unless stated otherwise, elsewhere in the present disclosure. Further, in some embodiments, one or more users may be required to successfully perform authentication in order for the control input to be effective. In general, a user of one or more users may perform authentication based on the possession of a secret human-readable secret data (e.g. username, password, passphrase, PIN, secret question, secret answer, etc.) and/or possession of a machine-readable secret data (e.g. encryption key, decryption key, bar codes, etc.) and/or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera, and so on. Likewise, the one or more steps may include receiving, using the communication device, the one or more embodied characteristics from one or more biometric sensors.

Further, one or more steps of the method may be automatically initiated, maintained, and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device, etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor, etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, one or more steps of the method may be performed one or more number of times. Additionally, one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data, and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Overview

The present disclosure describes methods, systems, apparatuses, and devices for facilitating monitoring and managing diseases of users.

Further, the present disclosure describes systems and methods for monitoring and managing respiratory diseases.

Further, the present disclosure describes an app or a device connecting to the cloud that helps the patient and a platform that helps physicians remotely manage their patients. The app operates by connecting to a respiratory device and receiving respiratory data for a patient from the device, receiving a list of medications and an action plan, tracking medications usage and adherence, tracking objective measures including (but not limited to) peak flow, FEV1, FEV6, FVC, and SpO2, sharing a remote patient monitoring report with a healthcare professional, providing a personalized chatbot and two-way messaging between the patient and healthcare professional, and tracking symptoms, triggers, and reporting medication usage.

Further, the device connecting to the cloud platform directly or through an intermediate data hub is also an option if the patient is unable to use the app. In this scenario, the patient gets text messages without PII as reminders while also alerting the physicians or monitoring team of any escalations.

Implementations of the system may include one or more of the following. The system can provide a real-time allergen report. The system provides an educational portal where patients can learn about health issues with resources in the portal. Advantages of the system may include one or more of the following. The digital platform creates personalized care experiences for patients. The app allows patients to monitor and self-manage their respiratory health. The system communicates with connected devices to easily collect and track patient health. Patients can maintain transparency with their physicians through automated remote patient monitoring (RPM) reports. The platform helps with monitoring and improving patients' health outcomes by providing feedback and empowering the patient to manage their condition. With the system, patient and physician engagement is made easy through an evidence-based tool (EBT), which helps patients make the best decisions by allowing them to enter their symptoms and providing personalized care recommendations. The system reduces costs for the patient, providers, and payers. The system helps the user in managing his or her respiratory health. If the inhaled medicine is used incorrectly, it won't help the user. The system provides reminders on usage, and periodically checks that the medication is used properly. The system can provide custom instructions, depending on the types of bronchodilators. Some people with asthma or COPD need treatment with inhaled corticosteroids, which can curb swelling in the air passages. Some of these medications are short-acting. Others are long-acting. And some kick in quickly, while others take time to work. The system also aids the user in keeping track of the medication. Since it's so important to control COPD, the system can track how much medicine remains so refills can be done on time. For ILD/Pulmonary fibrosis the system allows tracking spirometry readings over time.

Another crucial part of the platform is the monitoring solution. The monitoring platform allows appropriate alerts and escalations to be sent to the monitoring team or physicians to ensure that the right patients are attended to without creating a significant overhead. Remotely monitoring their daily condition and responding early to changes helps signal an impending worsening of their symptoms. Physician burnout is a crucial issue and any solution that requires significant overhead to the physician's workflow is problematic. Providing crucial alerts and summary reports that ensure that the population is correctly managed is extremely important. The monitoring platform is used when the physician or monitoring team gets an alert regarding a patient or the physician wants to see how their patient population is behaving (i.e. which patients are in the red, yellow, or green zone). In the case of an alert or when the physician wants to get a better understanding of a patient's health and trends the physician or monitoring team member reviews an individual patient record. Further, they can review the health of the patient over the last 30 days by looking at how many red, yellow or green days the patient has reported or looking at any messages from the patient or the engine, or looking at nighttime/daytime medication usage and adherence to their regular medications.

Additionally, the physician looks at trends of data that are captured either through patient reports or connected devices for several metrics including but not limited to Peakflow (PEF), FEV1, FVC, FEV6, SPO2, and flow volume loops.

If the physician notices a problem or requires a follow-up from the patient, they can use the two-way messaging feature to send a message or direct the patient to an action they need to perform.

As the physician or monitoring team uses the platform, it automatically captures the time spent on the platform per patient. This information along with the data collected from the patient is used for reimbursement from payors.

Further, the present disclosure describes taking evidence-based guidelines, patient profiles as well as publicly available information to automatically generate executable rules for patient care. And during execution, these rules look at patient-specific data collected from various inputs like, but not limited to:
a. Environmental factors
b. Device & App Interactions
c. Publicly available information
d. Patient history from EHR, Claims data
e. Physician defined care plan (also known as Action Plan)
f. Other external systems such as data from other health devices or apps

Further, every interaction from the patient with the app or device triggers these rules. The evaluation of these rules provides a chat-like interaction with the patient. It also generates alerts or messages for the monitoring team as needed.

Further, the present disclosure describes a system for remote patient monitoring of respiratory diseases such as asthma and COPD in accordance with one embodiment. This implementation provides a system and method for managing respiratory diseases by providing remote patient monitoring and applying evidence based rules to manage the disease. The methods can include: a) Connecting to a respiratory device and receiving respiratory data for a patient from the device; b) Receiving a list of medications and an action plan; c) Tracking medication symptoms including peak flow and SpO2; d) Sharing a remote patient monitoring report with a healthcare professional, and e) Providing a personalized chatbot and two-way messaging between the patient.

In one embodiment, the patient has been diagnosed with asthma and has had a pulmonary function test (PFT) which is captured as part of their medical record. The patient downloads an app that handles the entire workflow process including registration, provisioning of equipment, and analyzing results.
A) Connecting to a respiratory device and receiving respiratory data for a patient from the device, wherein the respiratory data includes airflow volume of at least one breathing cycle;
B) Receiving, in response to connection with the respiratory device, action plan instructions configured to guide breathing therapy.

In one example, the patient can connect his or her respiratory device to the app and receive a device connection notification on his/her smartphone. The patient may then accept the device connection request from the app and his/her asthma controller will begin communicating with the app using any technology such as Bluetooth Low Energy (BLE) technology, which enables low-power wireless communications over short distances. Preferably, a respiratory device (a peak flow meter) is wirelessly attached to the mobile phone or the patient's device (an inhaler or any other portable device that stores medication).

The app connects to a respiratory device and receives respiratory data for a patient from the device. The app can be designed for mobile platforms such as iOS or Android or operating systems like Windows or MacOS. The platform supports various remote monitoring devices including Bluetooth-connected spirometers which can be used to track the patient's lung function over time and make it easier for physicians to optimize a patient's medications. The remote digital monitoring app provides deeper insights into a patient's progress than could be obtained over several in person office visits. For example, the app is compatible with a variety of devices and operating systems, for example, the Mir Spirobank and Andes Fit Oximeter, among others.

The device may be any portable medical device that collects data related to a patient's health. For example, the respiratory device may include a spirometer that collects data related to peak flow, FEV1, FEV6 (etc) and/or SpO2. Peak flow is measured in liters per minute (LPM) or a percentage of the predicted normal value. SpO2 is measured as a percentage (%). Patients with low SpO2 have difficulty delivering oxygen from their lungs to their bloodstream, which can result in serious complications. The app can also connect to a weighing scale, a pulse oximeter, a spirometer, an oxygen concentrator, or another breathing assistance apparatus.

The system can help the patient conform to a medication regimen prescribed by a physician. In some cases, a patient is instructed to complete a medication action plan that contains pre- and post-measurements and may include measurements taken throughout the day. The system can communicate with medication intake devices and assist patients with their medication intake.

In a disclosed example, the app can receive respiratory data from a respiration device of the patient. The app can communicate with the device using a plurality of types of communication protocols including those for transferring files, transmitting messages, and/or sending commands to or from the device.

The app tracks medications, physiologic parameters & symptoms. The physiologic readings are tracked including peak flow, FEV1, FEV6, and SpO2, for example. These are recorded as a parameter that is further used to track a report that can be sent to the doctor or a suitable healthcare professional along with other details like medications, allergies, etc. This report is then used by the rules executed by the system and supervised/controlled by a doctor for determining the diagnosis of an acute episode and whether it requires medication change or more immediate attention. Patients can log into their healthcare provider account via a browser and mobile app. The patient can record peak flow and SpO2 using a treatment device. Video communications can be done with a professional using a video camera, smartphone, or wearable device.

The user can use a mobile app to log in with their credentials. The user is then greeted by a welcome screen that directs them to a dashboard page, where they can view all their information. They have access to all their supported devices under their account. The device monitor provides a current snapshot of the user's vital signs and medication adherence in one location. The results screen shows the user's temperature, pulse rate, respiratory rate, blood pressure, oxygen saturation, and steps per day with detailed graphs showing these readings over time.

The system thus automates the patient diary to record their symptoms, medication, and triggers. Patients with chronic diseases like asthma need to track the peak flow (a measurement of how well the lungs can bring in the air) and blood oxygen levels. Tracking these measures helps physicians make treatment decisions for the patient. Patients can also report the effects of multiple medications to control their disease which makes it difficult to manage and may be associated with an increased risk of side effects.

Patients suffer from breathing disorders and often need to regularly manage their symptoms by monitoring medication usage. Medication administration is time-consuming, often involving counting the number of pills to be taken at regular intervals throughout the day. Some medications have side effects that the patient may need to monitor. To provide assistance, the system receives a list of medications and an action plan. The action plan may include self-care, assessment, or reporting symptoms, selecting severity level, monitoring health data (e.g., peak flow), providing a report to a healthcare professional (HCP), asking for a second opinion, and prescribing medication from the physician's collection. The patient can choose an action on the list by clicking on it.

The present disclosure describes the action plan generated by the system. The action plan is a personalized schedule for taking medications to control the respiratory symptoms and maximize compliance. An example of an action plan can be:

As a patient, the user can check in daily, enter her symptoms and triggers and view her peak flow chart. One can also track medications, learn about disease management, and connect with her provider.

Medications can be managed by adding or removing medications from the app.

The app allows the user to either call a physician or an emergency number.

With the app, the user can perform the following:

Connect with patients' physicians, access patients' care plans, and get personalized tips at any minute and any day.

With daily check-ins and continuous feedback, the app ensure constant monitoring of patients' symptoms and triggers.

Information on the use of inhalers and medications can improve outcomes and help in disease management.

General training as suggested by the patients physician.

Get a report of patients' medication and symptoms, then share it with patients' physician to create patients' personalized care plan.

The system breaks down barriers to the exchange of information and insights between patients and health experts. The system provides education on a digital platform that connects patients and physicians remotely. Advanced medical insights need not be limited to the people residing in the most popular or wealthy zip codes. While there is no present replacement for a full in-person physical examination by a medical professional, digital networks are pivotal in narrowing the gap in accessing health literacy. Furthermore, the quantity and fidelity of data that can now be collected at home and sent securely to the doctor's office have improved by leaps and bounds with the development of medical devices which allow for remote patient monitoring. The instant smartphone application connects patients with asthma and other respiratory conditions with their physicians. From the comfort of their home, patients can share their symptoms, questions, and progress with medical professionals and make improvements in their health.

From the physician's perspective, the platform provides a way to ensure that there is a way to manage and monitor patients with respiratory diseases.

Further, the present disclosure describes an architecture of a mobile application (“app”) to monitor and manage patient respiratory issues. The mobile app includes a health advisor that communicates with the health platform. The app has a view, view model/controller, and data access modules that communicate with a local data store. Security, configuration and push notifications can be handled by the app leveraging the platform where needed.

Further, the present disclosure describes a cloud-based system to monitor and manage patient respiratory issues. All references to AWS below can be replaced by equivalent technologies on platforms like Azure, GCP and Oracle cloud. Further, the system includes the following:

Application services:
There are three applications deployed in the cloud. One is an application for API services and two are applications for the Web based User Interface.
The application logs can be stored in a solution like AWS Cloudwatch.
The application cluster is deployed using highly available technology such as AWS Fargate. It runs multiple replicas of the services in different availability zones.
For the mobile clients, Android and IOS applications and a responsive web browser are developed.

For patients without a mobile device, a browser on a desktop can can be used.

Database:
The application uses a database that can be based on either a relational model such as AWS RDS or a NoSQL model.
The storage encryption is enabled for the database server. The encryption keys are stored in a cloud based solution such as AWS KMS itself. The keys are self managed by platform.
The database server set up is highly available with a standby instance configured in a different availability zone.
Automated backups are enabled for the database server and point time recovery is also configured.
The database logs are stored using a a service such as AWS Cloudwatch.

Object Storage:
The files/images are stored in private S3 buckets.
The S3 objects can not be accessed directly using the object URL. In order to access the objects, a presigned URL is generated which is valid for a specific time period.

Caching:
The cache data is stored in caching technology such as AWS Elasticache (Redis).
The cluster is configured to run multiple instances of a solution like Redis for high availability.

Security:
A firewall such as Cloudflare is configured for all three applications.
Attacks like DDoS, XSS, Brute force login attempts, SQL Injections, DNS spoofing, etc. can be detected and prevented in Cloudflare.
The connection from the client(Web, Mobile) to the server is encrypted using SSL.
Encryption at rest is enabled.
The applications are configured to run inside a private network in VPC. These applications can not be reached directly from the internet. Only the load balancer can forward the traffic to the applications.
The database server also resides in the private network in a VPC. The database server can not be directly accessed from the internet. To access the database outside AWS VPC, a bastion host is used which acts like a proxy server. Code and Deployments:
The source code is stored in source control repositories.
The applications are configured to run as docker containers.
The docker images are stored in private repositories in a solution like AWS ECR.
In order to deploy the application, the bastion host is run. The docker images are built on the bastion host and pushed to the ECR repositories. The bastion is assigned AWS IAM role to perform this operation.
The bastion host is on-demand and it has limited access. Only whitelisted IP addresses can connect to the bastion host using ssh authentication.

Other Services:
SES is used for sending emails.
SNS is used for sending push notifications to mobile devices.
Twilio is used for sending SMS.
Pollen or Air Quality APIs are used to get air index.

Further, the present disclosure describes a method for monitoring and communicating between patients and physicians. In the method, an engine receives patient data/history, evidence paper/knowledge base, and external factors such as environment, location, and allergen). The engine receives patient data via a Talk module. The talk module receives data from connected devices and mobile devices, among others. The talk module generates actions, messages, recommendations, and stores device reading data. The physician receives alerts, messages, EMR history, and treatment options through a platform module in communication with the engine.

Further, the present disclosure describes a process for generating rules for the engine. The process starts with acquiring patient action, patient history, medical evidence, clinic/physician workflow, and external factors such as allergens and location data. The process generates rules for patient care and follows up by deriving medication rules, follow rules, healthcare maintenance rules, escalation management rules, and reminders. The rules are reviewed and applied once approved. Further, such rules can be mapped from government recommendations, for example from the CDC or GINA asthma guidelines.

For example, the system can conform to guidelines-based medical management by presenting information to patients such as:
Receive training in delivering guidelines-based medical management of asthma or COPD.
Review inhaler technique (if applicable) with patients with asthma, their families, and their caregivers, which can be reinforced with CDC's videos on “Know How to Use Your Asthma Inhaler.”
Use decision-support tools (such as treatment algorithms, system reminders, and pocket-sized guidelines summaries) and shared decision-making (in which healthcare professionals work with patients to decide on treatment) when caring for patients with asthma.
Participate in quality improvement activities to improve care for patients with asthma. Some health departments have asthma control programs that can be partners in healthcare quality improvement for patients with asthma.
Refer patients with asthma, their families, and their caregivers to available support services (when applicable) such as prescription assistance programs that offer reduced costs for prescription medications.
Use a patient-centered medical home model (which prioritizes coordinated care) to deliver primary care.
Improve communication and coordination with caregivers and schools about caring for children with asthma (for example, by creating and sharing asthma action plans).

In one embodiment, the system teaches patients with asthma, their families, and their caregivers to manage asthma by using a personalized action plan through the app.

The system can show information to patients with asthma, their families, and their caregivers about existing asthma self-management education (AS-ME) programs. The system can routinely screen:
Patients with asthma for tobacco use and exposure to secondhand smoke.
Family members and caregivers of patients with asthma for tobacco use.
Provide guideline-based cessation treatment including behavioral counseling (individual, group, or telephone based) and cessation medications approved by the U.S. Food and Drug Administration to patients with asthma, family members, and caregivers who use tobacco.
Connect patients with asthma, family members, and caregivers who use tobacco to community tobacco cessation services, including state guidelines for more intensive assistance.

Further, the present disclosure describes rules for managing patient respiratory issues. For example, one set of pseudo code for rules can be:

```
if patient checks in YELLOW
  capture symptoms
  if patient took Quick Relief
    Ask “Are you feeling better? Yes or No”
      If “Yes”
        Reply “Ok. Great Message”
        Check who they are doing after 4 hrs
      else
        Show Inhaler Video
  else if patient did not take Quick Relief
    Ask “Why didn't you take quick relief?”
      Capture reasons
    If reason is related to side effect
      Send message to physician
```

In one implementation of a process for escalating patient care, the pseudo-code is as follows:
Escalation
POS “Spirometry17.new current green no symptoms”
See Also: POS “Spirometry19.new current yellow or red no symptoms” (De-Escalation)
4.1.6.2
GROUP_10:20
4.1.1.2, 4.1.2.1, 4.1.3.1, 4.1.4.1 fired today
keva.msg.to.patient.4.1.6.2=That seems to have worked because the peak flow you just reported is within your normal range. Good job!
POS “Spirometry19.new current yellow or red no symptoms”
See Also: POS “Spirometry17.new current green no symptoms” (De-Escalation)
NOT “KEVA.ATHOME112.y/r/e or sx 0-15 min ago”
4.1.1.2
NOT “keva.athome.4.1.1.2_0-0 days”,“KEVA.ATHOME112.y/r/e or sx 0-15 min ago” (4.1.1.2 NOT fired today)
GROUP_10:19
4.1.1.2 did not fire before today AND
(No y/r/e checkins OR No symptoms in last 15 mins)
keva.msg.to.patient.4.1.1.2=The peak flow you just entered was low for you. Can you recheck it? Do 3 measurements and then click here and enter the highest of the 3 results.
PeakFlow
4.1.2.1
NOT “keva.athome.4.1.2.1_0-0 days”, (4.1.2.1 NOT fired today)
GROUP_10:18
4.1.2.1 did not fire before today AND
(No y/r/e checkins OR No symptoms in last 15 mins)
keva.msg.to.patient.4.1.2.1=That peak flow was still low. Please try again. Remember: stand up straight and take the deepest breath you can. Close your lips tightly around the mouthpiece and keep your tongue away from the mouthpiece. Then blow out as hard and as quickly as possible until you have emptied out nearly all of the air from your lungs.
PeakFlow
4.1.3.1
NOT “keva.athome.4.1.3.1_0-0 days”,“KEVA.ATHOME112.y/r/e or sx 0-15 min ago” (4.1.3.1 NOT fired today)
GROUP 10:17
4.1.3.1 did not fire before today AND
(No y/r/e checkins OR No symptoms in last 15 mins)
keva.msg.to.patient.4.1.3.1=I'm puzzled as to why your Peak Flow is so low. Can you watch this 2 minute video on how to check your peak flow and then try again?
VideoPeakflowUsage
4.1.4.1
NOT “keva.athome.4.1.4.1_0-0 days” (4.1.4.1 NOT fired today)
GROUP_10:16
4.1.4.1 did not fire before today AND
(No y/r/e checkins OR No symptoms in last 15 mins)
keva.msg.to.patient.4.1.4.1=Your peak flow is still low for you. Maybe your meter is dirty. Have you cleaned it recently?
reply yn
Yes
keva.msg.to.patient.4.1.4.1.y=Then I am NOT sure why your peak flow is low. At this point a discussion might be a more efficient way to figure this out. Would you like someone to call you?
No
keva.msg.to.patient.4.1.4.1.y.n=Ok. If you change your mind you can always send a text from the bottom of this messages screen.
Yes
keva.msg.to.patient.4.1.4.1.y.y=Great. I'll have someone get in touch with you soon.
keva.msg.to.dr.4.1.4.1.y.y=Patient had 4th low Peak Flow. Has tried repeating, standing and other technique tips, video, and says has cleaned it recently. Would like to talk to a clinician to try and figure it out. Maybe the goal NOT right? Call them to troubleshoot.
Physician Msg
No
keva.msg.to.patient.4.1.4.1.n=You can use hot water and a mild detergent unless it is an electronic device. Can you clean it and then try to measure your peak flow one more time?
PeakFlow
4.1.5.1
NOT keva.athome.4.1.5.1_0-0 days”, “keva.athome.4.1.5.1 y in last 2 wks” (4.1.5.1 NOT fired today, and NO 4.1.5.1_y NOT fired in last 2 weeks)
GROUP 10:15
4.1.5.1 did not fire before today AND
(No yellow/red/emergency checkins OR No symptoms in last 15 mins)
keva.msg.to.patient.4.1.5.1=We have gone through the most common causes of low peak flow in the absence of symptoms. Would you like someone to call you to help figure this out?
reply yn
Yes
Patient
keva.msg.to.patient.4.1.5.1.y=Great. I'll have someone get in touch with you soon.
MD
keva.msg.to.dr.4.1.5.1.y=Patient had 5th low Peak Flow. Has tried repeating, standing and other technique tips, video, and cleaning it. Would like to talk to a clinician to try and figure out. Maybe goal NOT right? Call them to troubleshoot.
Physician Msg
No
keva.msg.to.patient.4.1.5.1.n=Ok. If you change your mind you can always send a text from the bottom of this messages screen.
POS “KEVA.ATHOME133.new yellow check in”
keva.msg.to.dr.4.2.1.4=Patient checked in yellow.
Physician Msg
POS “KEVA.ATHOME94.new red checkin”,“KEVA.ATHOME95.new emerg checkin”
4.4.6.1
keva.msg.to.patient.4.4.6.1=This app does NOT manage patients who are critically ill. If your asthma is ‘red’ or ‘emergency’ please talk to a provider immediately or call an ambulance.
TEST NOTE
keva.msg.to.dr.4.4.6.1=This app does NOT manage patients who are critically ill. If your asthma is ‘red’ please talk to a provider immediately or call an ambulance.
Physician Msg
POS “KEVA.ATHOME96.yellow checkin 5-10 min ago”
4.2.1.1
NOT “keva.athome.4.2.1.1_0-10 min” (4.2.1.1 did NOT fire in last 10 mins)
NOT “KEVA.ATHOME125.mild in last 20 min”,“KEVA.ATHOME126.mod in last 20 min”,“KEVA.ATHOME127.sev in last 20 min”
keva.msg.to.patient.4.2.1.1=You checked in yellow a couple minutes ago but didn't enter any symptoms. Can you tell me a bit about the symptoms you were having?
Enter Symptoms
POS “KEVA.ATHOME97.new mild symptom”,“KEVA.ATHOME98.new moderate symptom”,“KEVA.ATHOME99.new severe symptom”
4.2.3.1
NOT KEVA.MED129.PRN asthma meds taken in last hour”
keva.msg.to.patient.4.2.3.1=Did you take quick relief medication already for your symptoms?
Yes
keva.msg.to.patient.4.2.3.1.y=Please update the total number of doses you have taken today on the medication page by tapping ‘reply’.
No
keva.msg.to.patient.4.2.3.1.n=Do you plan on taking your quick relief medication now?
No
keva.msg.to.patient.4.2.3.1.n.n=Do you have your quick relief with you?
Yes
keva.msg.to.patient.4.2.3.1.n.n.y=Can you tell me why you are NOT planning on using your quick relief medication for your symptoms?
Free Text
No
keva.msg.to.patient.4.2.3.1.n.n.n=Will you be able to get to your quick relief shortly?
Yes
keva.msg.to.patient.4.2.3.1.n.n.n.y=Ok. Let me know if your symptoms get worse. Once you have taken your quick relief, click here to report how many doses.
No
keva.msg.to.patient.4.2.3.1.n.n.n.n=Do you want to go to the student health center and get some quick relief medicaiton there?
Yes
Patient
keva.msg.to.patient.4.2.3.1.n.n.n.n.y=Ok. I'll let them know you are on your way. Please check in again once you get there so I know you are okay.
MD
keva.msg.to.dr.4.2.3.1.n.n.n.n.y=Patient going to student health center to get some quick relief meds as don't have their quick relief with them and can't get to it soon.
No
Patient
keva.msg.to.patient.4.2.3.1.n.n.n.n.n=Ok. I'll check back with you in a little while to see if your symptoms are getting better on their own. If you think your asthma is being triggered by cold air try to move indoors. Or if you think your asthma is being triggered by indoor allergens, try to move to a different location.
MD
keva.msg.to.dr.4.2.3.1.n.n.n.n.n=Patient having symptoms, doesn't have QR with them, can't get to it shortly, and doesn't want to go to health center. Check back with patient in a few minutes to see if doing any better.
Yes
keva.msg.to.patient.4.2.3.1.n.y=Once you've taken your quick relief, click here or go to the medications page to report how many doses you took.
4.2.3.2i
POS KEVA.MED129.PRN asthma meds taken in last hour”
keva.msg.to.patient.4.2.3.2=Did the quick relief you took help with your symptoms?
No
No 1
KEVA.MED59.has a spacer”
keva.msg.to.patient.4.2.3.2.n1=Did you use your spacer?
Yes
keva.msg.to.patient.4.2.3.2.n1.y=Do you think you might need to take more puffs of quick relief right now?
NOT “keva.athome.4.2.3.2.n1.y.y1_0-0 days” (if 4.2.3.2.n1.y.y1 NOT fired today)
Yes
Patient
Yes 1
keva.msg.to.patient.4.2.3.2.n1.y.y1=Ok. Once you have taken your meds, click here to let me know how many puffs you took.
Yes 2
keva.msg.to.patient.4.2.3.2.n1.y.y2=It seems like you are needing an unusual amount of quick relief medication today. Do you want to talk to a doctor about this?
Yes
Patient
keva.msg.to.patient.4.2.3.2.n1.y.y2.y=Ok. I'll have someone get in touch with you soon and meanwhile, if you still think you need more quick relief you should take it.
MD
keva.msg.to.dr.4.2.3.2.n1.y.y2.y=Patient having symptoms, took quick relief, didn't help, used the spacer, thinks they need more quick relief, NOT the first time today they answered this. want to talk to a clinician about the unusual amount of quick relief needing today (and meanwhile told them to take more quick relief if they think they need it)
No
Patient
keva.msg.to.patient.4.2.3.2.n1.y.y2.n=Ok. If you feel you need more quick relief you should take it, and we can address this again if you are still NOT getting adequate relief from your quick relief. Once you have taken your meds, click here to let me know how many puffs you took.
MD
keva.msg.to.dr.4.2.3.2.n1.y.y2.n=Patient having symptoms, took quick relief, didn't help, used the spacer, thinks they need more quick relief, NOT the first time today they answered this. they declined to talk to a clinician about the unusual amount of quick relief needing today so told them to take more quick relief if they think they need it and let us know how much. follow up with patient if haven't heard from them in a few minutes.
MD
keva.msg.to.dr.4.2.3.2.n1.y.y1=Patient having symptoms, took quick relief, didn't help, used the spacer, thinks they need more quick relief, first time today they answered that, advised to try that and then check in again. Follow up with patient if they don't check in soon.
No
No 1
keva.msg.to.patient.4.2.3.2.n1.y.n1=Ok. Let's keep tracking your symptoms and we can address this again if you continue to have sub-optimal relief from your quick relief medication.
No 2
keva.msg.to.patient.4.2.3.2.n1.y.n2=You seem to be having frequent problems with your quick relief NOT working adequately. Do you want to talk to a clinician about this?
Yes
Patient
keva.msg.to.patient.4.2.3.2.n1.y.n2.y=I'll have someone get in touch with you soon.
MD
keva.msg.to.dr.4.2.3.2.n1.y.n2.y=Patient having symptoms, took quick relief, didn't help, used the spacer, doesn't think they need more quick relief, and this has happened already more than once in past 2 weeks. Patient wants to talk to a clinician about problems with quick relief NOT working adequately. Call patient now.
No
keva.msg.to.patient.4.2.3.2.n1.y.n2.n=Ok. If you change your mind you can always send a text from the bottom of this messages screen.
MD
keva.msg.to.patient.4.2.3.2.n1.y.d=Do you want to talk to a clinician about whether or NOT you should take more quick relief now?
Yes
Patient
keva.msg.to.patient.4.2.3.2.n1.y.d.y=Ok. I'll have someone get in touch with you soon and meanwhile, if your breathing is bad and you think you need more quick relief you should take it.
MD
keva.msg.to.dr.4.2.3.2.n1.y.d.y=Patient having symptoms, took quick relief, didn't help, used the spacer, earlier they had same issue and thought they needed more quick relief. now doesn't know if they need more quick relief. would like to talk to a clinician about the unusual amount of quick relief needing today. call patient
No
Patient
keva.msg.to.patient.4.2.3.2.n1.y.d.n=Ok. If you change your mind you can send a secure text message from the bottom of this screen or call your clinician. Please check in again in a little while and let me know how you are feeling.
MD
keva.msg.to.dr.4.2.3.2.n1.y.d.n=Patient having symptoms, took quick relief, didn't help, used the spacer, thought they needed more quick relief, now NOT sure if need even more quick relief. declined to talk to a clinician about the unusual amount of quick relief needing today. follow up with patient if haven't heard from them in a few minutes.
spacer?
No
keva.msg.to.patient.4.2.3.2.n1.n=Can you tell me why you didn't use your
NOTe: Free Text
No 2
4.2.3.2.n2
KEVA.MED59.has a spacer”
keva.msg.to.patient.4.2.3.2.n2=Usually people who feel like their quick relief meds aren't working well benefit from review of their inhaler technique. Would you like to watch a video reviewing inhaler technique?
Yes
keva.msg.to.patient.4.2.3.2.n2.y=Click here to watch a video reviewing inhaler technique (you can also get to this video when you check in yellow and then select ‘Tips’).
No
keva.msg.to.patient.4.2.3.2.n2.n=Would you like to take a 10 second video of yourself taking your inhaler and text it to me?
Yes
Patient
keva.msg.to.patient.4.2.3.2.n2.n.y=We are still building out this feature. For now, if you know how, you can take a video using your phone's camera and text it to Virtual Clinician (617 444 9094).
MD
keva.msg.to.dr.4.2.3.2.n2.n.y=Patient having symptoms, took quick relief, didn't help, doesn't have a spacer, didn't want to watch a video but said they would like to take a selfie video of themselves using inhaler and text to us. Look out for the video.
Physician Msg
No
keva.msg.to.patient.4.2.3.2.n2.n.n=How about considering a spacer (an aerochamber). Is this something you would like to learn more about?
Yes
Patient
keva.msg.to.patient.4.2.3.2.n2.n.n.y=Great. I'll have someone get in touch with you soon.
MD
keva.msg.to.dr.4.2.3.2.n2.n.n.y=Patient having symptoms, took quick relief, didn't help, doesn't have a spacer but would like to learn more about spacers. Text patient with info about spacers/aerochamber.
Physician Msg
No
No 1
4.2.3.2.n2.n.n.n1
GROUP_13:2
NOT “keva.athome.4.2.3.2.n2.n.n.n1_0-14 days”,(NOT fired in last 14 days)
keva.msg.to.patient.4.2.3.2.n2.n.n.n1=Ok. Let's keep tracking your symptoms and we can address this again if you continue to have inadequate relief from your quick relief medication.
No 2
4.2.3.2.n2.n.n.n2
GROUP_13:1
keva.msg.to.patient.4.2.3.2.n2.n.n.n2=You seem to be having frequent problems with your quick relief NOT working adequately. Do you want to talk to a clinician about this?
No
keva.msg.to.patient.4.2.3.2.n2.n.n.n2.n=Ok. If you change your mind you can always send a text from the bottom of this messages screen.
Yes
Patient
keva.msg.to.patient.4.2.3.2.n2.n.n.n2.y=Great. I'll have someone get in touch with you soon.
MD
keva.msg.to.dr.4.2.3.2.n2.n.n.n2.y=Patient having symptoms, took quick relief, didn't help, doesn't have a spacer, doesn't want to learn more about spacers. More than 1st time had this situation in past 2 weeks, wants to talk to a clinician about their frequent problems with quick relief NOT working adequately.
Physician Msg
Yes
keva.msg.to.patient.4.2.3.2.y=Great. Please check in again in a little while to let me know how you are feeling.
POS KEVA.MED100.new PRN asthma meds taken”
3.3.1.1
NOT “KEVA.ATHOME135.mild in last 60 min”,“KEVA.ATHOME136.mod in last 60 min”,“KEVA.ATHOME137.sev in last 60 min”
keva.msg.to.patient.3.3.1.1=Were you having symptoms when you used your quick relief?
Yes
keva.msg.to.patient.3.3.1.1.y=Please tell me what symptoms you were having.
No
keva.msg.to.patient.3.3.1.1.n=Was it before exercise that you used your quick relief?
Yes
keva.msg.to.patient.3.3.1.1.n.y=After using the quick relief, did you feel like you were able to exercise to your full potential?
No
keva.msg.to.patient.3.3.1.1.n.y.n=Did you take your quick relief inhaler at least 15 minutes before you started exercising?
No
keva.msg.to.patient.3.3.1.1.n.y.n.n=Ok. Try taking your quick relief at least 15 minutes before you start exercising next time and let me know how that goes.
Yes
keva.msg.to.patient.3.3.1.1.n.y.n.y=Taking your quick relief at least 15 minutes before exercising is good. Here are some other tips to help with exercised-induced asthma: 1) Warm-up for at least 10 minutes, 2) Gradually increase the intensity of your exercise, 3) Avoid exercising in extreme temperatures or when the air quality is poor, 4) When exercising in the cold, cover your mouth and nose to warm the air.
yes
Yes1
3.3.1.1.n.y.y1
GROUP_14:2
NOT “keva.athome.3.3.1.1.n.y.y_0-60 days” (NOT fired in last 60 days)
keva.msg.to.patient.3.3.1.1.n.y.y1=Great. If you don't already do this, you should keep some quick relief in your sneakers and/or in your gym bag so you always remember to take it at least 15 minutes before you start exercising.
Yes 2
3.3.1.1.n.y.y2
GROUP_14:1
keva.msg.to.patient.3.3.1.1.n.y.y2=Great. Glad to hear. Keep logging your quick relief whenever you use it so we can use the data to optimize your management on an ongoing basis.
No
keva.msg.to.patient.3.3.1.1.n.n=Was it before exposure to aNOTher trigger that you used your quick relief?
Yes
keva.msg.to.patient.3.3.1.1.n.n.y=Did you feel like the quick-relief worked well to prevent your symptoms?
No
No2
NOT KEVA.MED59.has a spacer”
keva.msg.to.patient.3.3.1.1.n.n.y.n2=Usually people who feel like their quick relief meds aren't working well benefit from review of their inhaler technique. Would you like to watch a video reviewing inhaler technique?
No
keva.msg.to.patient.3.3.1.1.n.n.y.n2.n=Would you like to take a 10 second video of yourself taking your inhaler and text it to me?
No
keva.msg.to.patient.3.3.1.1.n.n.y.n2.n.n=How about considering a spacer (an aerochamber). Is this something you would like to learn more about?
yes
Patient
keva.msg.to.patient.3.3.1.1.n.n.y.n2.n.n.y=Great. I'll have someone get in touch with you soon.
MD
keva.msg.to.dr.3.3.1.1.n.n.y.n2.n.n.y=Patient too QR before exposure to a non-exercise trigger and did NOT feel like it worked well to prevent symptoms. wants to consider a spacer.
No
No2
keva.msg.to.patient.3.3.1.1.n.n.y.n2.n.n.n2=You seem to be having frequent problems with your quick relief NOT working adequately. Do you want to talk to a clinician about this?
yes
Patient
keva.msg.to.patient.3.3.1.1.n.n.y.n2.n.n.n2.y=Great. I'll have someone get in touch with you soon.
MD
keva.msg.to.dr.3.3.1.1.n.n.y.n2.n.n.n2.y=Patient wants to talk to clinician about QR NOT working adequately. Does NOT want to consider a spacer. Didn't want to watch a video on inhaler technique or take a selfie. Took QR earlier to prevent non-exercise related symptoms and did NOT feel like the QR worked well to prevent the symptoms. Call or text.
No
keva.msg.to.patient.3.3.1.1.n.n.y.n2.n.n.n2.n=Ok. If you change your mind you can always send a text from the bottom of this messages screen.
No1
keva.msg.to.patient.3.3.1.1.n.n.y.n2.n.n.n1=Ok, you don't want to consider a spacer right now. Let's keep tracking your symptoms and we can address this again if you continue to have suboptimal relief from your quick relief medication.
yes
keva.msg.to.patient.3.3.1.1.n.n.y.n2.n.y=We are still building out this feature in our app, but meanwhile you can take a video using your phone's camera and text it to Virtual Clinician (617 444 9094).
yes
keva.msg.to.patient.3.3.1.1.n.n.y.n2.y=Click here to watch a video reviewing inhaler technique (you can also get to this video when you check in yellow and then click on ‘Tips’).
No1
POS KEVA.MED59.has a spacer”
keva.msg.to.patient.3.3.1.1.n.n.y.n1=Did you use your spacer?
yes
keva.msg.to.patient.3.3.1.1.n.n.y.n1.y=Do you think you might need a higher dose of quick-relief before exposure to certain triggers?
No
No2
keva.msg.to.patient.3.3.1.1.n.n.y.n1.y.n2=You seem to be having frequent problems with your quick relief NOT working adequately. Do you want to talk to a clinician about this?
No
keva.msg.to.patient.3.3.1.1.n.n.y.n1.y.n2.n=Ok. If you change your mind you can always send a text from the bottom of this messages screen.
yes
Patient
keva.msg.to.patient.3.3.1.1.n.n.y.n1.y.n2.y=Great. I'll have someone get in touch with you soon.
MD
keva.msg.to.dr.3.3.1.1.n.n.y.n1.y.n2.y=Patient took quick relief to prevent symptoms before exposure to a trigger other than exercise. They did NOT feel the quick relief worked well to prevent their symptoms, and they did use their spacer. They don't think they need a higher dose of quick relief before exposure to certain triggers, and this has happened more than once in past 2 weeks. they say they would like to talk to a clinician about quick relief NOT working adequately. call them.
No1
NOT “keva.athome.3.3.1.1.n.n.y.n1.y.n1_0-14 days” (NOT fired in last 14 days
keva.msg.to.patient.3.3.1.1.n.n.y.n1.y.n1=Ok. Let's keep tracking your symptoms and we can address this again if you continue to have sub-optimal relief from your quick relief medication.
yes
Patient
keva.msg.to.patient.3.3.1.1.n.n.y.n1.y.y=Ok. I'll ask a clinician to review your record and get back to you on that soon.
MD
keva.msg.to.dr.3.3.1.1.n.n.y.n1.y.y=Patient took quick relief to prevent symptoms before exposure to a trigger other than exercise. They did NOT feel the quick relief worked well to prevent their symptoms, and they did use their spacer. They think they might need a higher dose of quick relief before exposure to certain triggers. review their record and see if they need extra counseling about specific triggers and or a higher dose of quick relief.
No
keva.msg.to.patient.3.3.1.1.n.n.y.n1.n=Can you tell me why you didn't use your spacer?
medComplianceReport
Yes
keva.msg.to.patient.3.3.1.1.n.n.y.y=Great. Glad to hear. Keep logging your quick relief whenever you use it so we can use the data to optimize your management on an ongoing basis.
No
Patient
keva.msg.to.patient.3.3.1.1.n.n.n=I'm confused. Earlier you said you used your quick relief to prevent symptoms. Can you give me some more details on why you used your quick relief?
MD
keva.msg.to.dr.3.3.1.1.n.n.n=Patient took quick relief, no symptoms, said it was NOT before exercise that they used their quick relief, and NOT before exposure to aNOTher trigger. Asked them for more details on why used quick relief. Look for free text answer.
4.2.3.2ii
POS “KEVA.ATHOME135.mild in last 60 min”,“KEVA.ATHOME136.mod in last 60 min”,“KEVA.ATHOME137.sev in last 60 min”
keva.msg.to.patient.4.2.3.2=Did the quick relief you took help with your symptoms?
No
No 1
KEVA.MED59.has a spacer”
keva.msg.to.patient.4.2.3.2.n1=Did you use your spacer?
Yes
keva.msg.to.patient.4.2.3.2.n1.y=Do you think you might need to take more puffs of quick relief right now?
NOT “keva.athome.4.2.3.2.n1.y.y1_0-0 days” (if 4.2.3.2.n1.y.y1 NOT fired today)
Yes
Patient
Yes 1
keva.msg.to.patient.4.2.3.2.n1.y.y1=Ok. Once you have taken your meds, click here to let me know how many puffs you took.
Yes 2
keva.msg.to.patient.4.2.3.2.n1.y.y2=li seems like you are needing an unusual amount of quick relief medication today. Do you want to talk to a doctor about this?
Yes
Patient
keva.msg.to.patient.4.2.3.2.n1.y.y2.y=Ok. I'll have someone get in touch with you soon and meanwhile, if you still think you need more quick relief you should take it.
MD
keva.msg.to.dr.4.2.3.2.n1.y.y2.y=Patient having symptoms, took quick relief, didn't help, used the spacer, thinks they need more quick relief, NOT the first time today they answered this. want to talk to a clinician about the unusual amount of quick relief needing today (and meanwhile told them to take more quick relief if they think they need it)
No
Patient
keva.msg.to.patient.4.2.3.2.n1.y.y2.n=Ok. If you feel you need more quick relief you should take it, and we can address this again if you are still NOT getting adequate relief from your quick relief. Once you have taken your meds, click here to let me know how many puffs you took.
MD
keva.msg.to.dr.4.2.3.2.n1.y.y2.n=Patient having symptoms, took quick relief, didn't help, used the spacer, thinks they need more quick relief, NOT the first time today they answered this. they declined to talk to a clinician about the unusual amount of quick relief needing today so told them to take more quick relief if they think they need it and let us know how much. follow up with patient if haven't heard from them in a few minutes.
MD
keva.msg.to.dr.4.2.3.2.n1.y.y1=Patient having symptoms, took quick relief, didn't help, used the spacer, thinks they need more quick relief, first time today they answered that, advised to try that and then check in again. Follow up with patient if they don't check in soon.
No
No 1
keva.msg.to.patient.4.2.3.2.n1.y.n1=Ok. Let's keep tracking your symptoms and we can address this again if you continue to have sub-optimal relief from your quick relief medication.
No 2
keva.msg.to.patient.4.2.3.2.n1.y.n2=You seem to be having frequent problems with your quick relief NOT working adequately. Do you want to talk to a clinician about this?
Yes
Patient
keva.msg.to.patient.4.2.3.2.n1.y.n2.y=I'll have someone get in touch with you soon.
MD
keva.msg.to.dr.4.2.3.2.n1.y.n2.y=Patient having symptoms, took quick relief, didn't help, used the spacer, doesn't think they need more quick relief, and this has happened already more than once in past 2 weeks. Patient wants to talk to a clinician about problems with quick relief NOT working adequately. Call patient now.
No
keva.msg.to.patient.4.2.3.2.n1.y.n2.n=Ok. If you change your mind you can always send a text from the bottom of this messages screen.
D
keva.msg.to.patient.4.2.3.2.n1.y.d=Do you want to talk to a clinician about whether or NOT you should take more quick relief now?
Yes
Patient
keva.msg.to.patient.4.2.3.2.n1.y.d.y=Ok. I'll have someone get in touch with you soon and meanwhile, if your breathing is bad and you think you need more quick relief you should take it.
MD
keva.msg.to.dr.4.2.3.2.n1.y.d.y=Patient having symptoms, took quick relief, didn't help, used the spacer, earlier they had same issue and thought they needed more quick relief. now doesn't know if they need more quick relief. would like to talk to a clinician about the unusual amount of quick relief needing today. call patient
No
Patient
keva.msg.to.patient.4.2.3.2.n1.y.d.n=Ok. If you change your mind you can send a secure text message from the bottom of this screen or call your clinician. Please check in again in a little while and let me know how you are feeling.
MD
keva.msg.to.dr.4.2.3.2.n1.y.d.n=Patient having symptoms, took quick relief, didn't help, used the spacer, thought they needed more quick relief, now NOT sure if need even more quick relief. declined to talk to a clinician about the unusual amount of quick relief needing today. follow up with patient if haven't heard from them in a few minutes.
No
keva.msg.to.patient.4.2.3.2.n1.n=Can you tell me why you didn't use your spacer?
NOTe: Free Text
No 2
4.2.3.2.n2
KEVA.MED59.has a spacer”
keva.msg.to.patient.4.2.3.2.n2=Usually people who feel like their quick relief meds aren't working well benefit from review of their inhaler technique. Would you like to watch a video reviewing inhaler technique?
Yes
keva.msg.to.patient.4.2.3.2.n2.y=Click here to watch a video reviewing inhaler technique (you can also get to this video when you check in yellow and then select ‘Tips’).
No
keva.msg.to.patient.4.2.3.2.n2.n=Would you like to take a 10 second video of yourself taking your inhaler and text it to me?
Yes
Patient
keva.msg.to.patient.4.2.3.2.n2.n.y=We are still building out this feature. For now, if you know how, you can take a video using your phone's camera and text it to Virtual Clinician (617 444 9094).
MD
keva.msg.to.dr.4.2.3.2.n2.n.y=Patient having symptoms, took quick relief, didn't help, doesn't have a spacer, didn't want to watch a video but said they would like to take a selfie video of themselves using inhaler and text to us. Look out for the video.
Physician Msg
No
keva.msg.to.patient.4.2.3.2.n2.n.n=How about considering a spacer (an aerochamber). Is this something you would like to learn more about?
Yes
Patient
keva.msg.to.patient.4.2.3.2.n2.n.n.y=Great. I'll have someone get in touch with you soon.
MD
keva.msg.to.dr.4.2.3.2.n2.n.n.y=Patient having symptoms, took quick relief, didn't help, doesn't have a spacer but would like to learn more about spacers. Text patient with info about spacers/aerochamber.
Physician Msg
No
No 1
4.2.3.2.n2.n.n.n1
GROUP_13:2
NOT “keva.athome.4.2.3.2.n2.n.n.n1_0-14 days”,(NOT fired in last 14 days)
keva.msg.to.patient.4.2.3.2.n2.n.n.n1=Ok. Let's keep tracking your symptoms and we can address this again if you continue to have inadequate relief from your quick relief medication.
No 2
4.2.3.2.n2.n.n.n2
GROUP_13:1
keva.msg.to.patient.4.2.3.2.n2.n.n.n2=You seem to be having frequent problems with your quick relief NOT working adequately. Do you want to talk to a clinician about this?
No
keva.msg.to.patient.4.2.3.2.n2.n.n.n2.n=Ok. If you change your mind you can always send a text from the bottom of this messages screen.
Yes
Patient
keva.msg.to.patient.4.2.3.2.n2.n.n.n2.y=Great. I'll have someone get in touch with you soon.
MD
keva.msg.to.dr.4.2.3.2.n2.n.n.n2.y=Patient having symptoms, took quick relief, didn't help, doesn't have a spacer, doesn't want to learn more about spacers. More than 1st time had this situation in past 2 weeks, wants to talk to a clinician about their frequent problems with quick relief NOT working adequately.
Physician Msg
Yes
keva.msg.to.patient.4.2.3.2.y=Great. Please check in again in a little while to let me know how you are feeling.
Preventive
“KEVA.ATHOME100.new green checkin”
1.2.4.1
GROUP_9:500
NOT “keva.athome.1.2.4.1_0-365000 days” (1.2.4.1 NOT fired in 365 days)
keva.msg.to.patient.1.2.4.1=Welcome! Thanks for downloading this app and logging in. Any questions so far?
Yes
keva.msg.to.patient.1.2.4.1.y=Please share your questions/concerns/feedback so I can improve.
Click OK and Free text
keva.msg.to.patient.1.2.4.1.y.o=Thanks for your feedback. I'll work on that. Meanwhile, remember to check in daily, whenever you have symptoms, and whenever you use your quick relief medication. It's fast, fun, and will help keep your asthma under good control. OK?
Yes
keva.msg.to.patient.1.2.4.1.y.o.y=Excellent. FYI, you can send text messages to the on-call physician from the bottom of this screen. Go ahead, try it out!
No
keva.msg.to.patient.1.2.4.1.y.o.n=Please tell me which part did NOT sound OK. E.g.: ‘daily check in’, ‘checking in when I have symptoms’, ‘checking in whenever I use my quick relief medication’.
OK
keva.msg.to.patient.1.2.4.1.y.o.n.o=Thank you for your feedback. I'm going to have someone from our support staff review this and get back to you. Meanwhile, wanted to let you know that you can send text messages to the on-call physician from the bottom of this screen. Go ahead, try it out!
No
keva.msg.to.patient.1.2.4.1.n=Great! Remember to check in daily, whenever you have symptoms, and whenever you use your quick relief medication. It's fast, fun, and will help keep your asthma under good control. OK?
1.4.2.9
GROUP_9:498
NOT “keva.athome.1.4.2.9 u”,“keva.athome.1.4.2.9_0-7 days”,KEVA.MED84.any doses of PRN asthma meds taken in past week“(NOT Fired in last 7 days & No Unread Messages, No doses of PRN asthma meds taken in past week)
MED65.on PRN meds for asthma”
keva.msg.to.patient.1.4.2.9=Quick question: have you used your quick relief inhaler at all in the past week?
Yes
Patient
keva.msg.to.dr.1.4.2.9.y=Patient took quick relief in the past week but didn't report in real time. Asked for number of puffs and when. Add records.
MD
keva.msg.to.dr.1.4.2.9.y=Patient took quick relief in the past week but didn't report in real time. Asked for number of puffs and when. Add records.
No
No2
keva.msg.to.patient.1.4.2.9.n2=You had symptoms this past week, can you tell me why you didn't use your quick relief at the time?
medComplianceReport
1.4.2.9.n1
“KEVA.ATHOME135.y/r/e or sx 0-7 days ago” (NOT Fired in last 7 days & No Unread Messages)
No1
keva.msg.to.patient.1.4.2.9.n1=Did you have any symptoms in the past week?
Yes
keva.msg.to.patient.1.4.2.9.n1.y=Can you tell me why you didn't use your quick relief when you were having symptoms?
No
keva.msg.to.patient.1.4.2.9.n1.n=Ok, great! Will check back with you again soon.
1.4.2.8
GROUP_9:497
NOT “keva.athome.1.4.2.8 u”,“keva.athome.1.4.2.8_0-90 days” (NOT Fired in last 90 days & No Unread Messages, No PRN meds for asthma taken)
NOT KEVA.MED65.on PRN meds for asthma”
keva.msg.to.patient.1.4.2.8=I don't have a quick relief medication on record for you. Do you have one?
Yes
keva.msg.to.patient.1.4.2.8.y=Great. Please tap ‘reply’ to add it to your medication list.
No
keva.msg.to.patient.1.4.2.8.n=Everyone with asthma should have a quick relief medication to take when their symptoms act up. Have you ever been on a medication like this?
Yes
keva.msg.to.patient.1.4.2.8.n.y=Ok. Can you tell me why you stopped?
medCompliance
No
keva.msg.to.patient.1.4.2.8.n.n=Do you think you need a quick relief inhaler?
yes
Patient
keva.msg.to.patient.1.4.2.8.n.n.y=Ok, I'll have a clinician review your records and see if they think a quick relief prescription is indicated. They should get back to you soon.
MD
keva.msg.to.dr.1.4.2.8.n.n.y=Patient thinks they need a quick relief inhaler. They don't have one and say they have never been on one.
No
Patient
keva.msg.to.patient.1.4.2.8.n.n.n=Can you tell me why you don't think you need a quick relief medication? (e.g.: infrequent symptoms, they don't work, too expensive)
MD
keva.msg.to.dr.1.4.2.8.n.n.n=Patient NOT on a quick relief, says has never been on one, doesn't think they need one, asked why NOT. Look for free text answer.
1.4.1.1
GROUP_9:495
NOT “keva.athome.1.4.1.1 u”,“keva.athome.1.4.1.1_0-90 days” (1.4.1.1 NOT Fired in last 90 days & No Unread Messages)
keva.msg.to.patient.1.4.1.1=Health Care Maintenance Task: Please review your medication list and make sure it is up to date. Use buttons on the bottom of the medication screen to navigate between the remove, fix and add medication screens.
removeMeds
addMeds
fixMeds
1.4.2.6
GROUP_9:494
NOT “keva.athome.1.4.2.6 u”,“keva.athome.1.4.2.6_0-90 days” (1.4.2.6 NOT Fired in last 90 days & No Unread Messages)
keva.msg.to.patient.1.4.2.6=Health Care Maintenance Task: Please review your smoking status and make sure it is up to date.
Tobacco
1.4.2.7
GROUP_9:493
NOT “keva.athome.1.4.2.7 u”,“keva.athome.1.4.2.7_0-28 days” (NOT Fired in last 28 days & No Unread Messages)
keva.msg.to.patient.1.4.2.7=Health Care Maintenance Task: Please take 2 minutes to review your asthma control over the past few weeks. Your answers will help me optimize your
reply_ATAQ
1.4.2.4
GROUP_9:491
“Spirometry9.any range Peak Flow in last year”
NOT “keva.athome.1.4.2.4 u”,“keva.athome.1.4.2.4_0-7 days”,“Spirometry8.any range Peak Flow in last week” (NOT Fired in last 7 days & No Unread Messages)
keva.msg.to.patient.1.4.2.4=Reminder: check your peak flow at least once a week.
PeakFlow
1.4.2.2
GROUP_9:489
MED59.has a spacer”
POS KEVA.MED64.took inhaled med in past week”
NOT “keva.athome.1.4.2.2 u”,“keva.athome.1.4.2.2_0-7 days” (NOT Fired in last 7 days & No Unread Messages)
keva.msg.to.patient.1.4.2.2=Reminder: clean your spacer weekly (rinse with warm water and soap, and make sure to dry thoroughly).
1.4.2.1
GROUP_9:488
“Spirometry8.any range Peak Flow in last week”
NOT “keva.athome.1.4.2.1 u”,“keva.athome.1.4.2.1_0-7 days” (NOT Fired in last 7 days & No Unread Messages)
keva.msg.to.patient.1.4.2.1=Reminder: clean your peak flow meter weekly (rinse with warm water and soap, and make sure to dry thoroughly).
Reminders
1.1.2.2
GROUP_3:2
This is a unique rule that generates daily reminders for the year.
NOT “keva.athome.1.1.2.1_0-364 days” (NOT fired in last 364 days)
“KEVA.ATHOME84.start time 24 hrs ago”
“KEVA.ATHOME86.time pattern daily same time”
Generate a schedule to fire daily rules for the year
Reminders
1.2.2.1
GROUP_1:2
This is a unique rule that generates daily reminders for the day.
“keva.athome.1.2.2.1_0-1 days” (NOT fired in one day)
“KEVA.ATHOME79.any checkin in last 1 day”
Other Questions or Free text
8.1.1.1
keva.msg.to.patient.8.1.1.1=1′11 have a clinician review this and get back to you shortly. Meanwhile, if you need help urgently, don't wait for an answer to your text—call for help.
keva.msg.to.dr.8.1.1.1=Patient sent in a free text. Look for ‘patient waiting for’ message. Details should be in comment field.
Physician Msg
8.1.1.2 keva.msg.to.patient.8.1.1.2=I got your message. The last thing I want to point out right now is the white home button in the top right corner of this screen. If you tap that you can explore the rest of this app on your own. I'll send you aNOTher message next time you check in.
keva.msg.to.dr.8.1.1.2=Patient sent in first free text. Look for ‘patient waiting for’ message. The word ‘test’ should be in comment field. If anything else, acknowledge it and ask them to add our number to their contact list as ‘Virtual Clinician’.
Physician Msg
KEVA.MED106.new med issue other“,KEVA.MED94.new med issue mild sx”,KEVA.MED95.new med issue mod sx“,KEVA.MED96.new med issue sev sx”,KEVA.MED97.new med issue need new rx“,KEVA.MED98.new med issue couldn't get to store”,KEVA.MED98.new med issue couldn't get to store“,KEVA.MED99.new med issue need help wi copay”,KEVA.MED100.new med issue too expensive“,KEVA.MED101.new med issue not sure needed”,KEVA.MED102.new med issue did not think needed“,KEVA.MED103.new med issue was not working”,KEVA.MED104.new med issue started something else“,KEVA.MED105.new med issue not sure why stopped”,“KEVA.ATHOME149.forgot”,“KEVA.ATHOME136.new med issue mild sx”,“KEVA.ATHOME137.new med issue mod sx”,“KEVA.ATHOME138.new med issue sev sx”,“KEVA.ATHOME139.new med issue need new rx”,“KEVA.ATHOME140.new med issue couldn't get to store”,“KEVA.ATHOME141.new med issue need help wi copay”,“KEVA.ATHOME142.new med issue too expensive”,“KEVA.ATHOME143.new med issue not sure needed”,“KEVA.ATHOME144.new med issue did not think needed”,“KEVA.ATHOME145.new med issue was not working”,“KEVA.ATHOME146.new med issue started something else”,“KEVA.ATHOME147.new med issue not sure why stopped”,“KEVA.ATHOME148.new med issue other”
keva.msg.to.dr.3.2.9.1=Patient having some issue with medication.
keva.msg.to.patient.3.2.9.1=Ok.

Further, the present disclosure describes a digital advisor or assistant who receives information from questions/free text. The advisor also receives reminder information and preventative data. The advisor executes rules that can activate and escalate the case to reduce harm to the patient.

The logic manages a patient through 3 different scenarios when they are doing well, when they are escalated and when they need reminders. When a patient is doing well, they need guidance to stay on track with their plan.

When a patient is escalated, they are asked to go through a series of steps to guide them through using an action plan, reporting their readings with the devices and providing guidance on their ideal ranges, or providing just-in-time education as needed.

When a patient is doing well the engine sends appropriate messages to guide the user through steps they need to take to continue maintaining their health.

The patient also gets reminders to take their medications as well as follow up reminders when they are escalated.

In each of these scenarios, the monitoring team or physicians platform also gets alerts through the engine. These alerts provide indicators of the patients' health based on thresholds set previously based on input from the physician.

Another, innovative part of our model is that the pseudo code listed above can be generated based on evidence-based guidelines. The information from medical articles and journals is uploaded to extract key entities that determine what the guidelines should be.

The system breaks down barriers to the exchange of information and insights between patients and health experts. The system provides education on a digital platform that connects patients and physicians remotely. Advanced medical insights need not be limited to the people residing in the most popular or wealthy zip codes. While there is no present replacement for a full in-person physical examination by a medical professional, digital networks are pivotal in narrowing the gap in accessing health literacy. Furthermore, the quantity and fidelity of data that can now be collected at home and sent securely to the doctor's office have improved by leaps and bounds with the development of medical devices which allow for remote patient monitoring. The instant smartphone application connects patients with asthma and other respiratory conditions with their physicians. From the comfort of their home, patients can share their symptoms, questions, and progress with medical professionals and make improvements in their health. Twenty-five million Americans grapple with asthma, one of many chronic illnesses that disproportionately impacts financially strained communities. The platform supports a growing list of remote monitoring devices, including Bluetooth-connected spirometers, which can track the patient's lung function over time and make it easier for physicians to optimize a patient's medications. It is possible that remote digital monitoring may eventually provide deeper insights into a patient's progress than could be obtained over several in person office visits.

The system allows patients to access and view data from their devices while away from home. This software platform also allows physicians to remotely monitor their patients using patients' existing devices. The receiving respiratory data from a device that the patient is wearing, be it connected to a computer, to a cellular network, or directly connected to the platform. The respiratory data can be transmitted by other devices including but not limited to mobile phones, tablets, etc. that can operate as wireless gateways.

Further, the present disclosure describes systems and methods with a platform including an application/website that helps the patient and physicians remotely manage their patients. This is done by connecting to a respiratory device and receiving respiratory data for a patient from the device; receiving a list of medications and an action plan; tracking medications usage and adherence; tracking objective measures including peak flow, FEV1, FEV6, FVC, and SpO2; sharing a remote patient monitoring report with a healthcare professional; providing a personalized chatbot and two-way messaging between the patient and healthcare professional; and tracking symptoms, triggers, and reporting medication usage

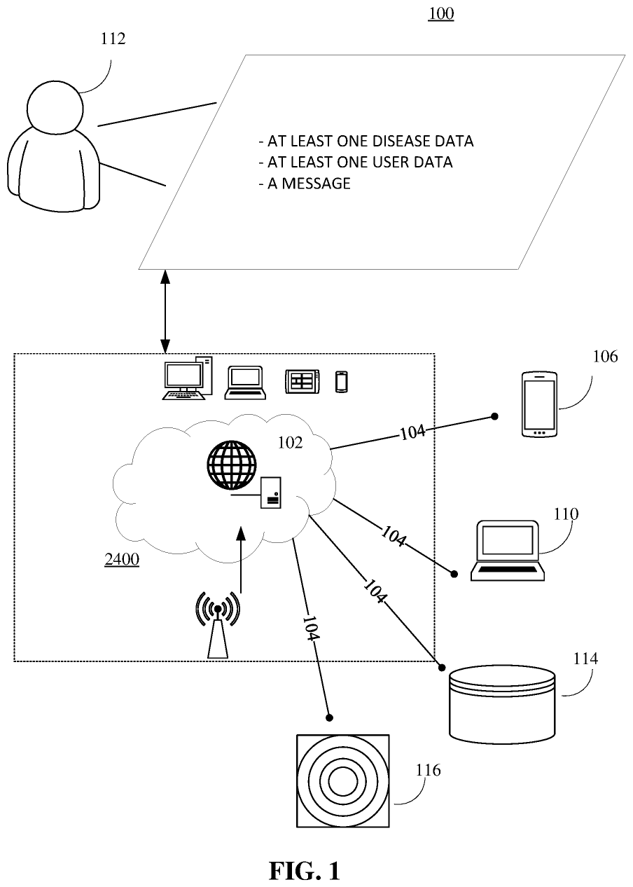

FIG. 1 is an illustration of an online platform 100 consistent with various embodiments of the present disclosure. By way of non-limiting example, the online platform 100 to facilitate monitoring and managing diseases of users may be hosted on a centralized server 102, such as, for example, a cloud computing service. The centralized server 102 may communicate with other network entities, such as, for example, a mobile device 106 (such as a smartphone, a laptop, a tablet computer, etc.), other electronic devices 110 (such as desktop computers, server computers, etc.), databases 114, and sensors 116 over a communication network 104, such as, but not limited to, the Internet. Further, users of the online platform 100 may include relevant parties such as, but not limited to, end-users, administrators, service providers, service consumers, and so on. Accordingly, in some instances, electronic devices operated by the one or more relevant parties may be in communication with the platform.

A user 112, such as the one or more relevant parties, may access online platform 100 through a web based software application or browser. The web based software application may be embodied as, for example, but not be limited to, a website, a web application, a desktop application, and a mobile application compatible with a computing device 2400.

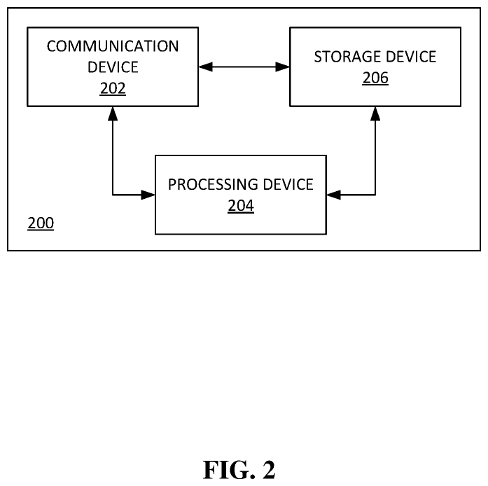

FIG. 2 is a block diagram of a system 200 for facilitating monitoring and managing diseases of users, in accordance with some embodiments. Accordingly, the system 200 may include a communication device 202, a processing device 204, and a storage device 206.

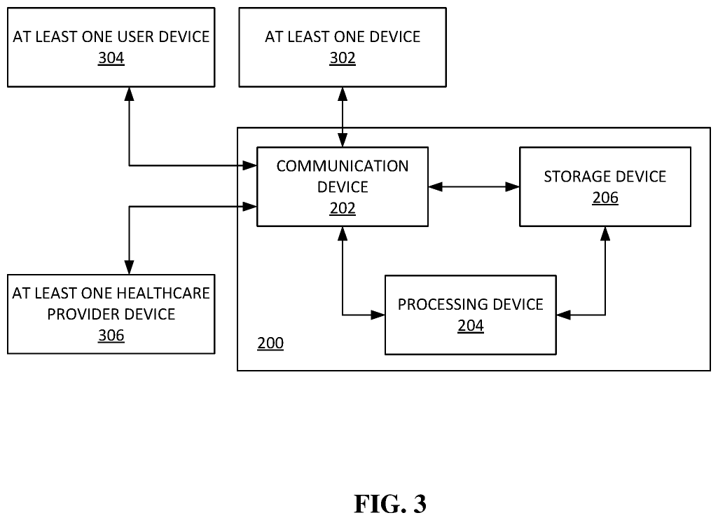

Further, the communication device 202 may be configured for receiving at least one disease data associated with at least one disease of at least one user from at least one device 302, as shown in FIG. 3. Further, the at least one user may include an individual, a patient, a patient's relative, etc. Further, the at least one device 302 may include a computing device, a client device, etc. Further, the at least one disease may include a respiratory disease, a heart disease, a liver disease, a renal disease, a gastrointestinal disease, diabetes, etc. Further, the at least one disease data may include at least one of an action plan and a prescription for managing the at least one disease. Further, the prescription may include at least one medication for the managing of the at least one disease. Further, the communication device 202 may be configured for receiving at least one user data associated with the at least one user from the at least one device 302. Further, the at least one user data may include at least one health record of the at least one user. Further, the communication device 202 may be configured for transmitting a message to at least one of at least one user device 304, as shown in FIG. 3, associated with the at least one user and at least one healthcare provider device 306, as shown in FIG. 3, associated with at least one healthcare provider. Further, the at least one user device 304 and the at least one healthcare provider device 306 may include a computing device, a client device, a user device, etc.

Further, the processing device 204 may be communicatively coupled with the communication device 202. Further, the processing device 204 may be configured for analyzing the at least one disease data and the at least one user data. Further, in an embodiment, the analyzing of the at least one disease data and the at least one user data may include analyzing the at least one disease data and the at least one user data using at least one machine learning model. Further, the at least one machine learning model may be trained for providing a diagnosis of the at least one user. Further, the processing device 204 may be configured for generating a report of the at least one user based on the analyzing of the at least one disease data and the at least one user data. Further, the report may include a remote patient monitoring report of the at least one user. Further, the processing device 204 may be configured for processing the report using at least one executable rule corresponding to the at least one disease. Further, the at least one excutable rule may be configured to be executed on the report. Further, the processing device 204 may be configured for determining a status of the at least one user based on the processing of the report. Further, the status corresponds to three different scenarios. Further, the three scenarios may include when the at least one user is doing well, when the at least one user is escalated, and when the at least one user need reminders. Further, the processing device 204 may be configured for generating the message for at least one of the at least one user and the at least one healthcare provider of the at least one user based on the status. Further, the message may include a guidance for the managing of the at least one disease.

Further, the storage device 206 may be communicatively coupled with the processing device 204. Further, the storage device 206 may be configured for storing the message for at least one of the at least one user and the at least one healthcare provider.

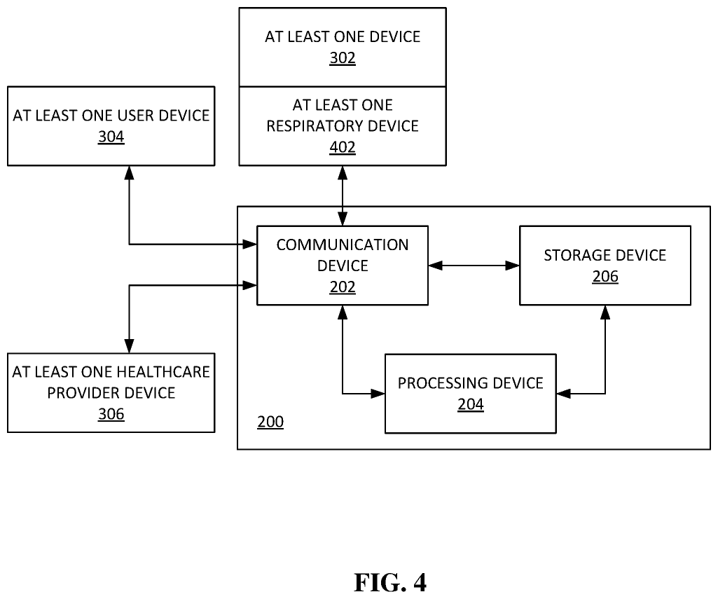

Further, in some embodiments, the at least one device 302 may include at least one respiratory device 402, as shown in FIG. 4. Further, the at least one respiratory device 402 may be configured for generating at least one respiratory data of the at least one user based on detecting at least one respiration characteristic of a respiration of the at least one user. Further, the at least one user data further may include the at least one respiratory data. Further, the at least one respiratory device 402 may include a spirometer, a pulse oximeter, etc. Further, the at least one respiratory data may include peak flow (PEF), FEV1, FVC, FEV6, SPO2, flow volume loops, etc. Further, the at least one respiration characteristic may include a respiratory rate, an inspiratory volume, a depth of respiration, a depth of chest movement, a chest expansion, a chest symmetry, a lung compliance, an airway resistance, etc.

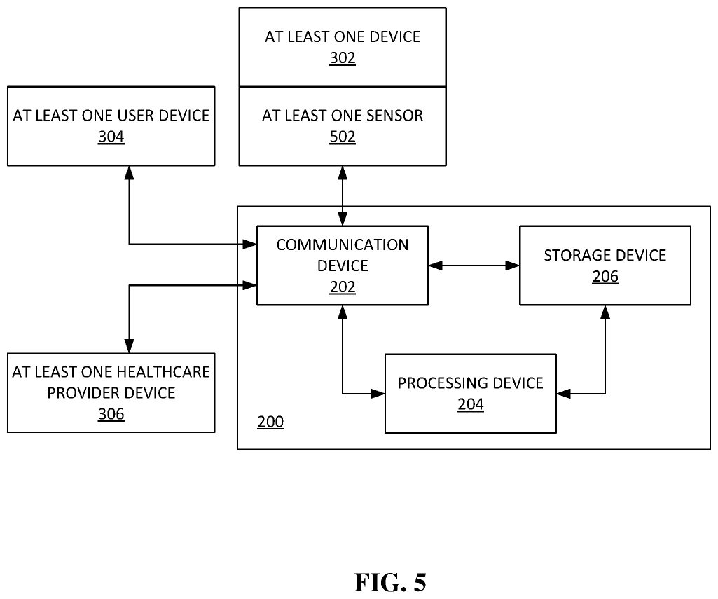

Further, in some embodiments, the at least one device 302 further may include at least one sensor 502 as shown in FIG. 5. Further, the at least one sensor 502 may be configured for generating at least one user measurement of the at least one user based on detecting at least one of a physical state, a physiological state, and a biological state of the at least one user. Further, the at least one user data further may include the at least one user measurement. Further, the at least one sensor may include a physical sensor, a physiological sensor, a biological sensor, etc. Further, the physical state may include a body weight, a body temperature, etc. Further, the physiological state may include a heartbeat rate, a blood pressure, SpO2, a pulse rate, a blood oxygen level, etc. Further, the biological state may include a level of one or more body hormones.

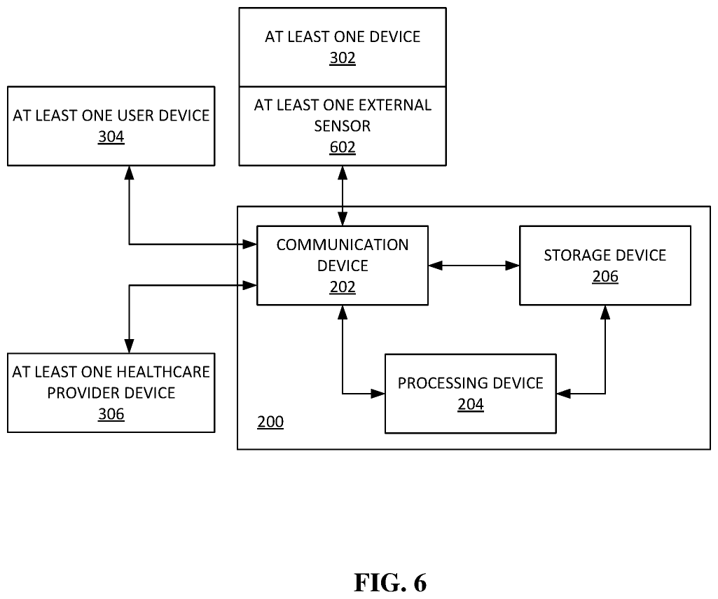

Further, in some embodiments, the at least one device 302 further may include at least one external sensor 602 as shown in FIG. 6. Further, the at least one external sensor 602 may be configured for generating at least one external sensor data associated with the at least one user based on detecting at least one external factor associated with the at least one user. Further, the at least one user data further may include the at least one external sensor data. Further, the at least one external factor may include an environmental factor, a location, an allergen, etc. associated with the at least one user. Further, the environmental factor may include an environmental temperature, an environmental pressure, an environmental humidity, etc.

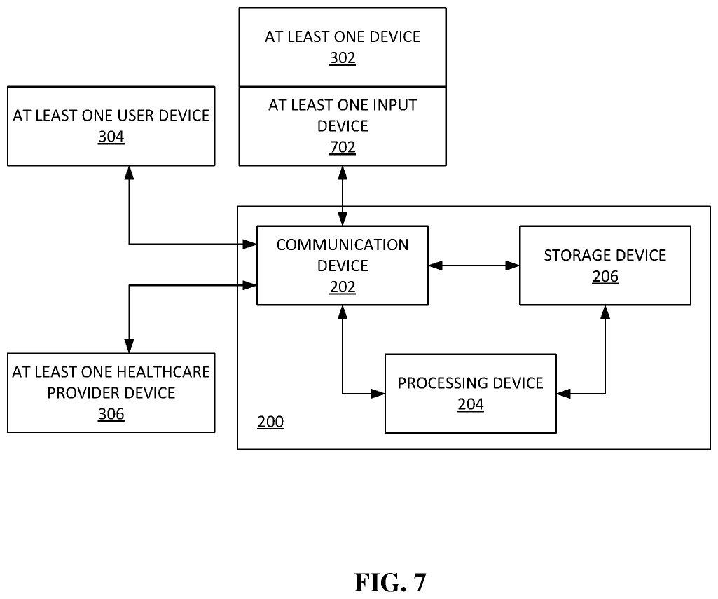

Further, in some embodiments, the at least one device 302 further may include at least one input device 702, as shown in FIG. 7. Further, the at least one input device 702 may be configured for receiving at least one assessment of the at least one user from the at least one user. Further, the at least one user data further may include the at least one assessment of the at least one user. Further, the at least one assessment may include symptoms, triggers, medication usage, etc.

Further, in some embodiments, the at least one disease data may include at least one identifier of the at least one disease. Further, the at least one identifier may include a name of the at least one disease. Further, the storage device 206 may be further configured for retrieving at least one data associated with the managing of the at least one disease based on the at least one identifier. Further, the processing device 204 may be further configured for analyzing the at least one data. Further, the generating of the report may be further based on the analyzing of the at least one data. Further, the at least one data may include evidence paper/knowledge base of the at least one disease.

Further, in some embodiments, the communication device 202 may be configured for transmitting the report to the at least one healthcare provider device 306. Further, the communication device 202 may be configured for receiving at least one remark of the at least one healthcare provider corresponding to the report from the at least one healthcare provider device 306. Further, the processing of the report may include processing the report and the at least one remark using the at least one executable rule. Further, the determining of the status may be further based on the processing of the report and the at least one remark. Further, the at least one remark may include a comment, a recommendation, a suggestion, etc. of the at least one healthcare provider.

Further, in some embodiments, the communication device 202 may be configured for transmitting the report to the at least one healthcare provider device 306. Further, the communication device 202 may be configured for receiving at least one remark of the at least one healthcare provider corresponding to the report from the at least one healthcare provider device 306. Further, the processing device 204 may be configured for initiating an interaction session between the at least one user and the at least one healthcare provider based on the at least one remark. Further, the processing device 204 may be configured for extracting at least one interaction between the at least one user and the at least one healthcare provider during the interaction session based on the initiating. Further, the processing of the report may include processing the report and the at least one interaction using the at least one executable rule. Further, the determining of the status may be based on the processing of the report and the at least one interaction. Further, the at least one interaction may include a communication between the at least one user and the at least one healthcare provider.

Further, in some embodiments, the at least one disease data may include at least one identifier of the at least one disease. Further, the processing device 204 may be configured for identifying at least one guideline of at least one medical authority for the managing of the at least one disease based on the at least one identifier. Further, the processing device 204 may be configured for analyzing the at least one guideline. Further, the processing device 204 may be configured for generating the at least one executable rule corresponding to the at least one disease based on the analyzing of the at least one guideline. Further, the processing of the report using the at least one executable rule may be based on the generating of the at least one executable rule.

FIG. 3 is a block diagram of the system 200, in accordance with some embodiments.

FIG. 4 is a block diagram of the system 200, in accordance with some embodiments.

FIG. 5 is a block diagram of the system 200, in accordance with some embodiments.

FIG. 6 is a block diagram of the system 200, in accordance with some embodiments.

FIG. 7 is a block diagram of the system 200, in accordance with some embodiments.

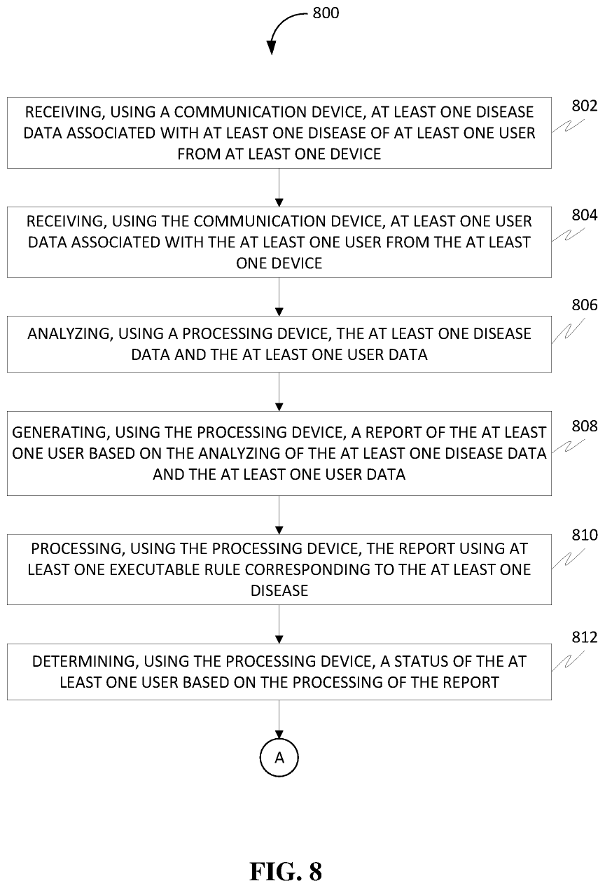

FIG. 8 is a flowchart of a method 800 for facilitating monitoring and managing diseases of users, in accordance with some embodiments. Accordingly, at 802, the method 800 may include a step of receiving, using a communication device, at least one disease data associated with at least one disease of at least one user from at least one device. Further, the at least one disease data may include at least one of an action plan and a prescription for managing the at least one disease.

Further, at 804, the method 800 may include a step of receiving, using the communication device, at least one user data associated with the at least one user from the at least one device. Further, the at least one user data may include at least one health record of the at least one user.

Further, at 806, the method 800 may include a step of analyzing, using a processing device, the at least one disease data and the at least one user data.

Further, at 808, the method 800 may include a step of generating, using the processing device, a report of the at least one user based on the analyzing of the at least one disease data and the at least one user data.

Further, at 810, the method 800 may include a step of processing, using the processing device, the report using at least one executable rule corresponding to the at least one disease.

Further, at 812, the method 800 may include a step of determining, using the processing device, a status of the at least one user based on the processing of the report.

Further, at 814, the method 800 may include a step of generating, using the processing device, a message for at least one of the at least one user and at least one healthcare provider of the at least one user based on the status. Further, the message may include a guidance for the managing of the at least one disease.

Further, at 816, the method 800 may include a step of transmitting, using the communication device, the message to at least one of at least one user device associated with the at least one user and at least one healthcare provider device associated with the at least one healthcare provider.

Further, at 818, the method 800 may include a step of storing, using a storage device, the message for at least one of the at least one user and the at least one healthcare provider.

Further, in some embodiments, the at least one device may include at least one respiratory device. Further, the at least one respiratory device may be configured for generating at least one respiratory data of the at least one user based on detecting at least one respiration characteristic of a respiration of the at least one user. Further, the at least one user data further may include the at least one respiratory data.

Further, in some embodiments, the at least one device further may include at least one sensor. Further, the at least one sensor may be configured for generating at least one user measurement of the at least one user based on detecting at least one of a physical state, a physiological state, and a biological state of the at least one user. Further, the at least one user data further may include the at least one user measurement.

Further, in some embodiments, the at least one device further may include at least one external sensor. Further, the at least one external sensor may be configured for generating at least one external sensor data associated with the at least one user based on detecting at least one external factor associated with the at least one user. Further, the at least one user data further may include the at least one external sensor data.

Further, in some embodiments, the at least one device further may include at least one input device. Further, the at least one input device may be configured for receiving at least one assessment of the at least one user from the at least one user. Further, the at least one user data further may include the at least one assessment of the at least one user.

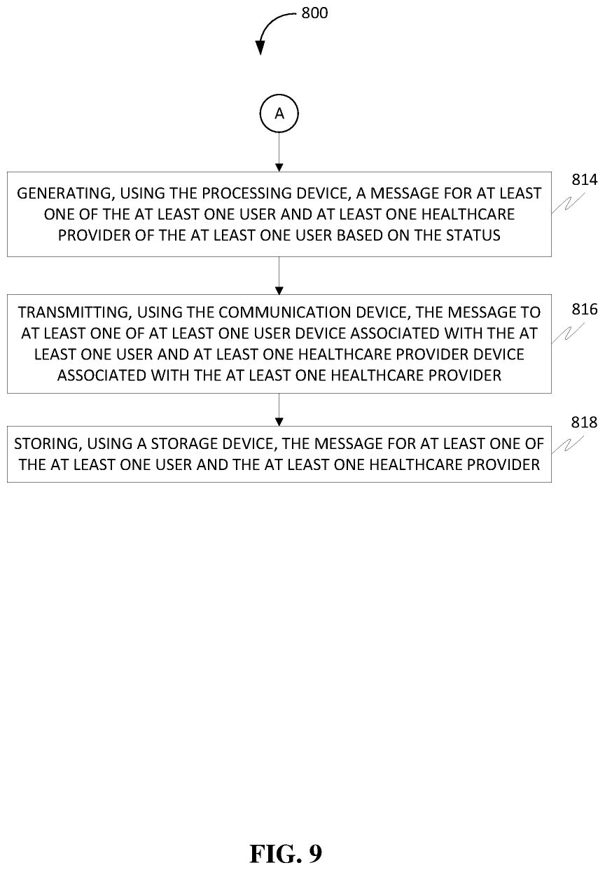

FIG. 9 is a continuation flowchart of the method 800 for facilitating the monitoring and managing of the diseases of the users, in accordance with some embodiments.

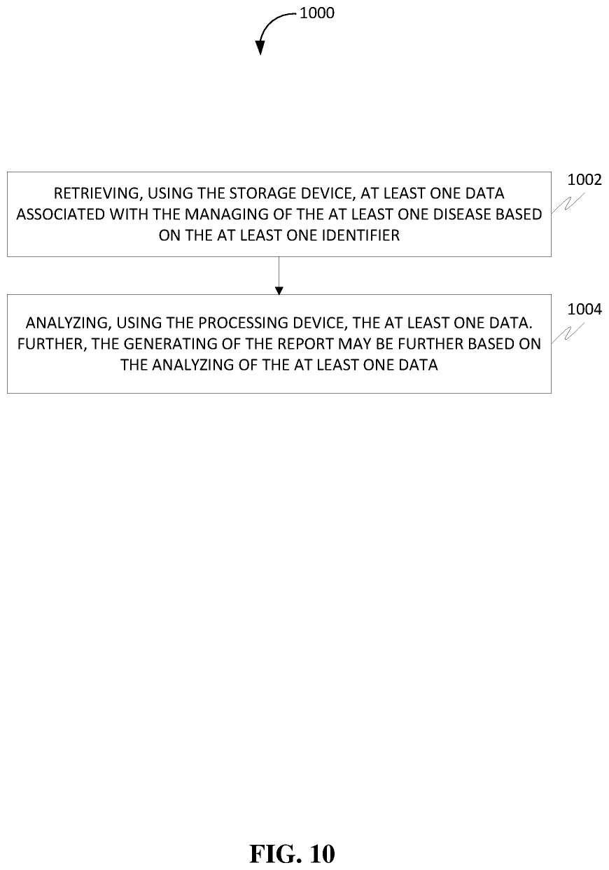

FIG. 10 is a flowchart of a method 1000 for retrieving data for facilitating the monitoring and managing of the diseases of the users, in accordance with some embodiments. Further, the at least one disease data may include at least one identifier of the at least one disease. Further, at 1002, the method 1000 may include a step of retrieving, using the storage device, at least one data associated with the managing of the at least one disease based on the at least one identifier.

Further, at 1004, the method 1000 may include a step of analyzing, using the processing device, the at least one data. Further, the generating of the report may be further based on the analyzing of the at least one data.

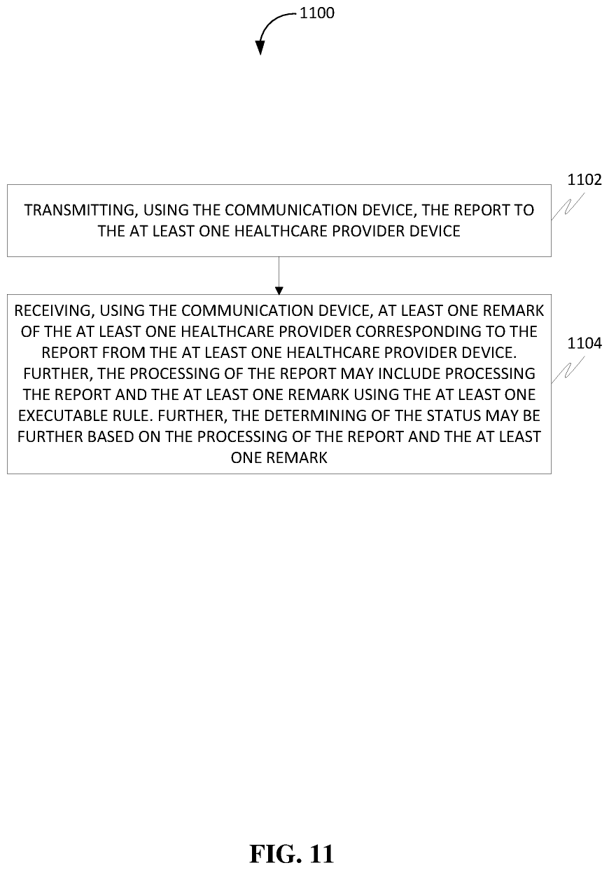

FIG. 11 is a flowchart of a method 1100 for determining the status based on remarks of healthcare providers for facilitating the monitoring and managing of the diseases of the users, in accordance with some embodiments. Further, at 1102, the method 1100 may include a step of transmitting, using the communication device, the report to the at least one healthcare provider device.

Further, at 1104, the method 1100 may include a step of receiving, using the communication device, at least one remark of the at least one healthcare provider corresponding to the report from the at least one healthcare provider device. Further, the processing of the report may include processing the report and the at least one remark using the at least one executable rule. Further, the determining of the status may be further based on the processing of the report and the at least one remark.

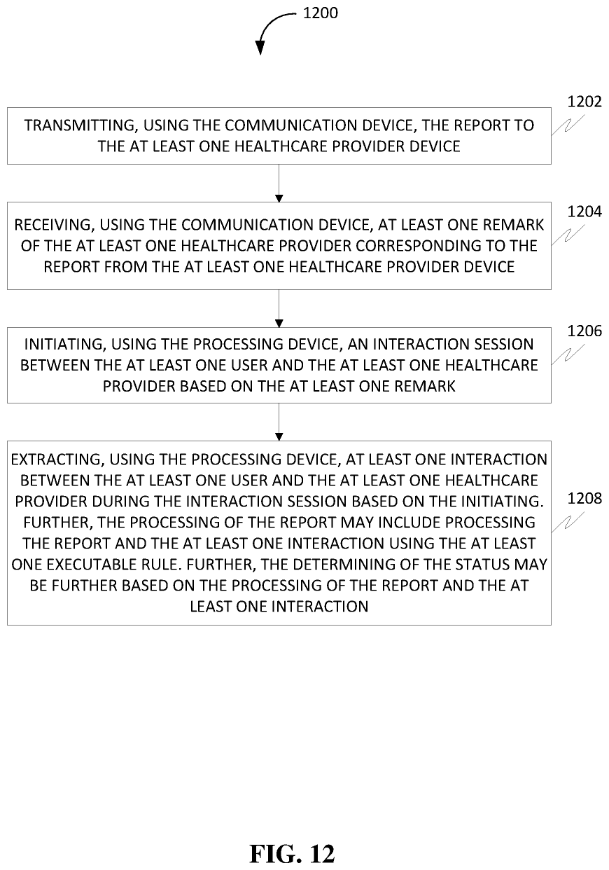

FIG. 12 is a flowchart of a method 1200 for determining the status based on interactions between the users and healthcare providers for facilitating the monitoring and managing of the diseases of the users, in accordance with some embodiments. Further, at 1202, the method 1200 may include a step of transmitting, using the communication device, the report to the at least one healthcare provider device.

Further, at 1204, the method 1200 may include a step of receiving, using the communication device, at least one remark of the at least one healthcare provider corresponding to the report from the at least one healthcare provider device.

Further, at 1206, the method 1200 may include a step of initiating, using the processing device, an interaction session between the at least one user and the at least one healthcare provider based on the at least one remark.

Further, at 1208, the method 1200 may include a step of extracting, using the processing device, at least one interaction between the at least one user and the at least one healthcare provider during the interaction session based on the initiating. Further, the processing of the report may include processing the report and the at least one interaction using the at least one executable rule. Further, the determining of the status may be based on the processing of the report and the at least one interaction.

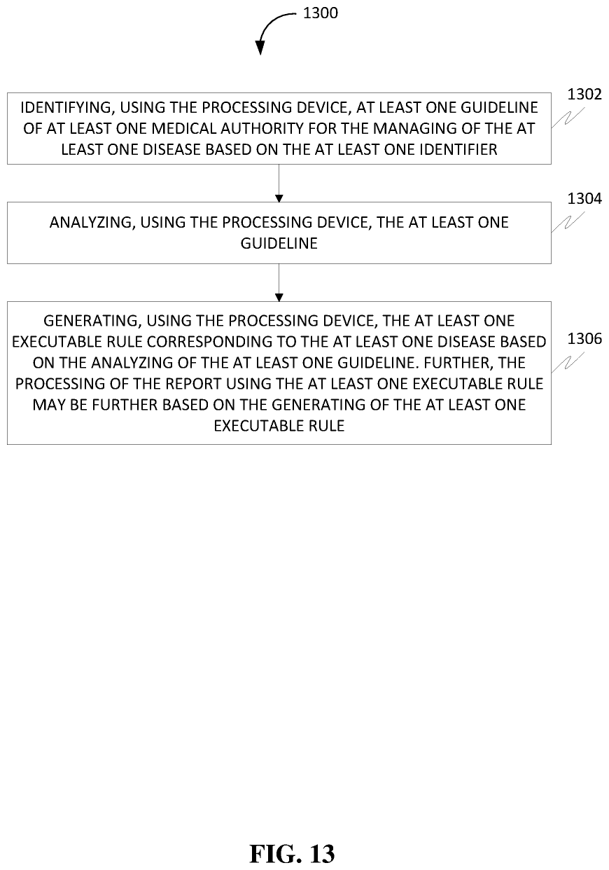

FIG. 13 is a flowchart of a method 1300 for generating executable rules for facilitating the monitoring and managing of the diseases of the users, in accordance with some embodiments. Further, the at least one disease data may include at least one identifier of the at least one disease. Further, at 1302, the method 1300 may include a step of identifying, using the processing device, at least one guideline of at least one medical authority for the managing of the at least one disease based on the at least one identifier.

Further, at 1304, the method 1300 may include a step of analyzing, using the processing device, the at least one guideline.

Further, at 1306, the method 1300 may include a step of generating, using the processing device, the at least one executable rule corresponding to the at least one disease based on the analyzing of the at least one guideline. Further, the processing of the report using the at least one executable rule may be based on the generating of the at least one executable rule.

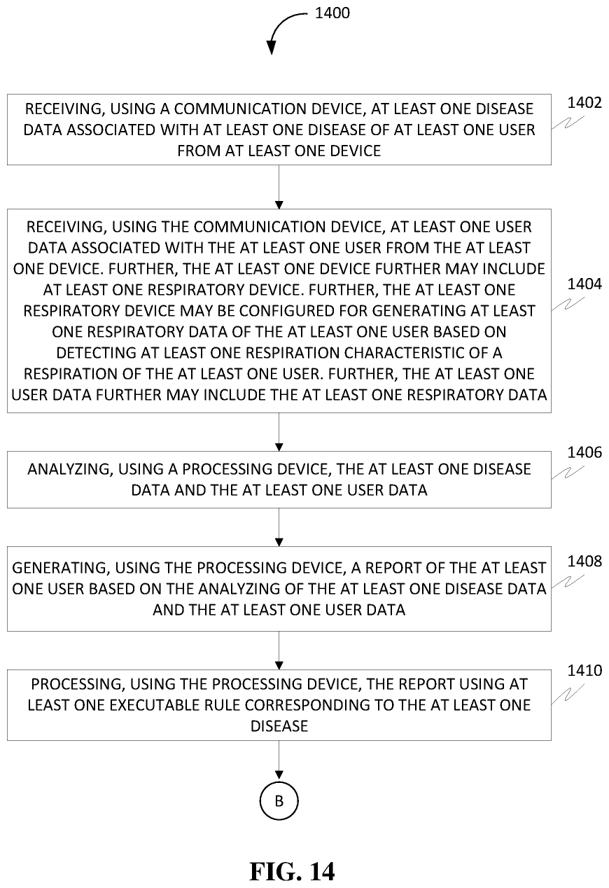

FIG. 14 is a flowchart of a method 1400 for facilitating monitoring and managing diseases of users, in accordance with some embodiments. Accordingly, at 1402, the method 1400 may include a step of receiving, using a communication device, at least one disease data associated with at least one disease of at least one user from at least one device. Further, the at least one disease data may include at least one of an action plan and a prescription for managing the at least one disease.

Further, at 1404, the method 1400 may include a step of receiving, using the communication device, at least one user data associated with the at least one user from the at least one device. Further, the at least one user data may include at least one health record of the at least one user. Further, the at least one device further may include at least one respiratory device. Further, the at least one respiratory device may be configured for generating at least one respiratory data of the at least one user based on detecting at least one respiration characteristic of a respiration of the at least one user. Further, the at least one user data further may include the at least one respiratory data.

Further, at 1406, the method 1400 may include a step of analyzing, using a processing device, the at least one disease data and the at least one user data.

Further, at 1408, the method 1400 may include a step of generating, using the processing device, a report of the at least one user based on the analyzing of the at least one disease data and the at least one user data.

Further, at 1410, the method 1400 may include a step of processing, using the processing device, the report using at least one executable rule corresponding to the at least one disease.

Further, at 1412, the method 1400 may include a step of determining, using the processing device, a status of the at least one user based on the processing of the report.

Further, at 1414, the method 1400 may include a step of generating, using the processing device, a message for at least one of the at least one user and at least one healthcare provider of the at least one user based on the status. Further, the message may include a guidance for the managing of the at least one disease.

Further, at 1416, the method 1400 may include a step of transmitting, using the communication device, the message to at least one of at least one user device associated with the at least one user and at least one healthcare provider device associated with the at least one healthcare provider.

Further, at 1418, the method 1400 may include a step of storing, using a storage device, the message for at least one of the at least one user and the at least one healthcare provider.

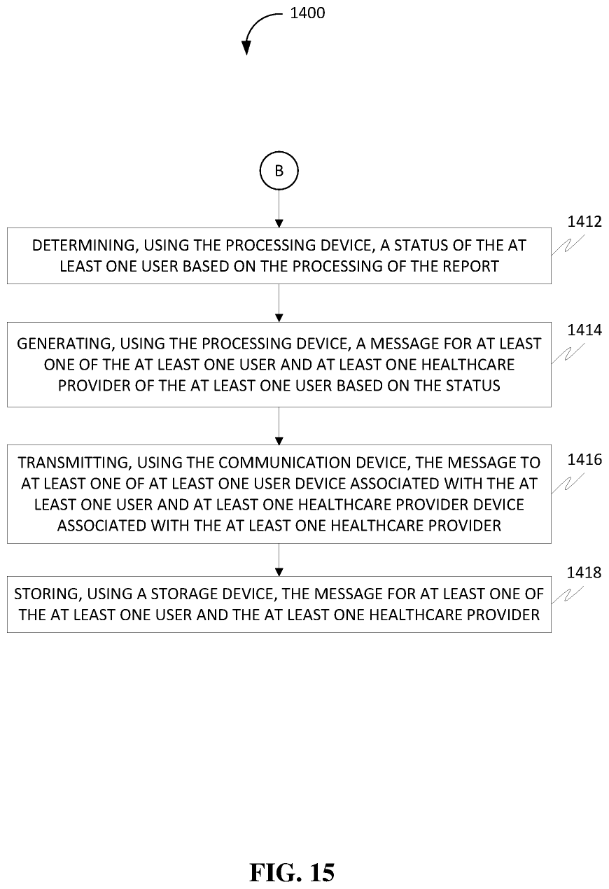

FIG. 15 is a continuation flowchart of the method 1400 for facilitating the monitoring and managing of the diseases of the users, in accordance with some embodiments.

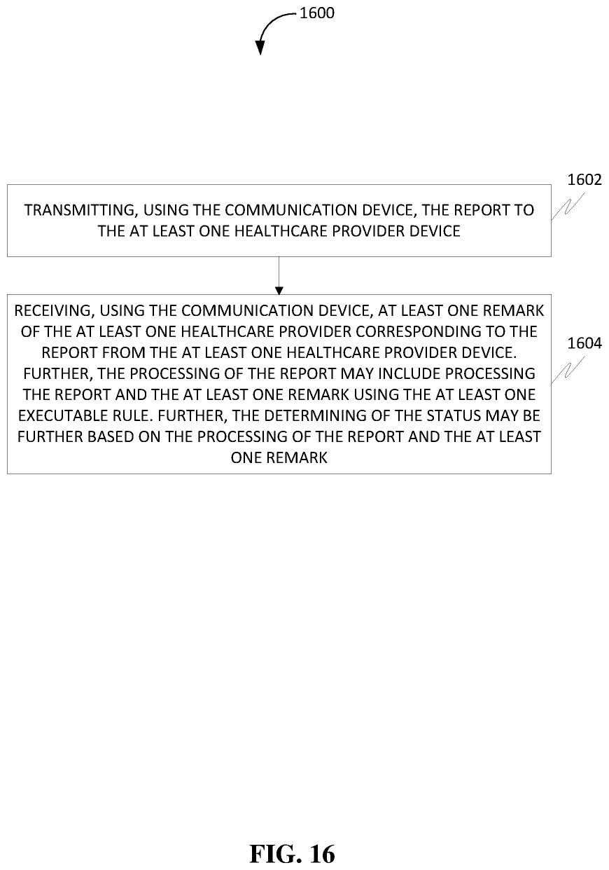

FIG. 16 is a flowchart of a method 1600 for determining the status based on remarks of healthcare providers for facilitating the monitoring and managing of the diseases of the users, in accordance with some embodiments. Further, at 1602, the method 1600 may include a step of transmitting, using the communication device, the report to the at least one healthcare provider device.

Further, at 1604, the method 1600 may include a step of receiving, using the communication device, at least one remark of the at least one healthcare provider corresponding to the report from the at least one healthcare provider device. Further, the processing of the report may include processing the report and the at least one remark using the at least one executable rule. Further, the determining of the status may be based on the processing of the report and the at least one remark.

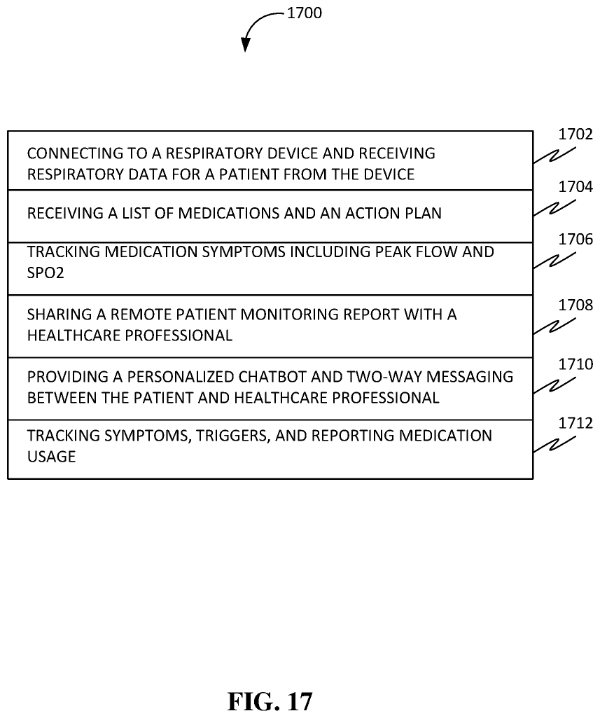

FIG. 17 is a flowchart of a method 1700 for facilitating monitoring and managing respiratory diseases of users, in accordance with some embodiments. Accordingly, at 1702, the method 1700 may include a step of connecting to a respiratory device and receiving respiratory data for a patient from the device.

Further, at 1704, the method 1700 may include a step of receiving a list of medications and an action plan.

Further, at 1706, the method 1700 may include a step of tracking medication symptoms including peak flow and SpO2.

Further, at 1708, the method 1700 may include a step of sharing a remote patient monitoring report with a healthcare professional.

Further, at 1710, the method 1700 may include a step of providing a personalized chatbot and two-way messaging between the patient and healthcare professional.

Further, at 1712, the method 1700 may include a step of tracking symptoms, triggers and reporting medication usage.

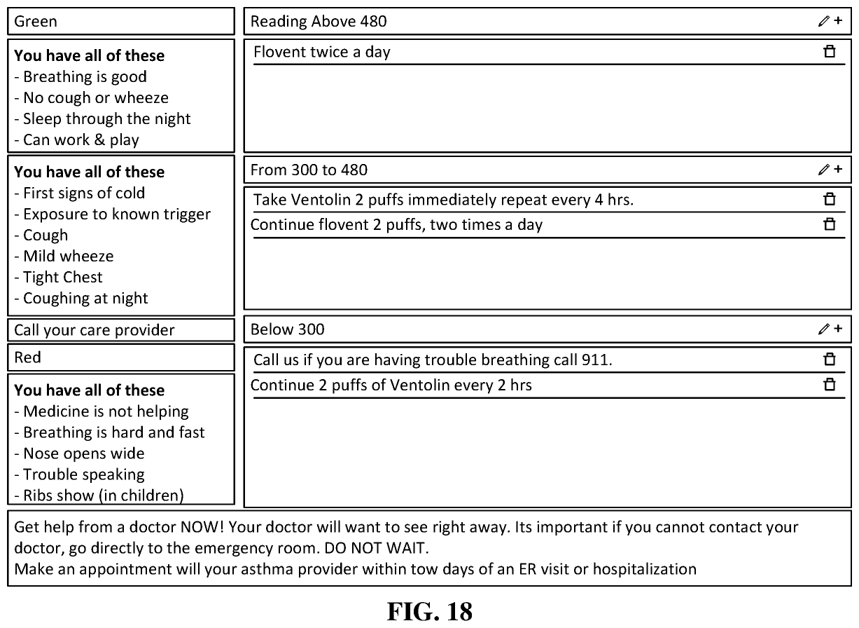

FIG. 18 illustrates an action plan for facilitating the monitoring and managing of the respiratory diseases of the users, in accordance with some embodiments.

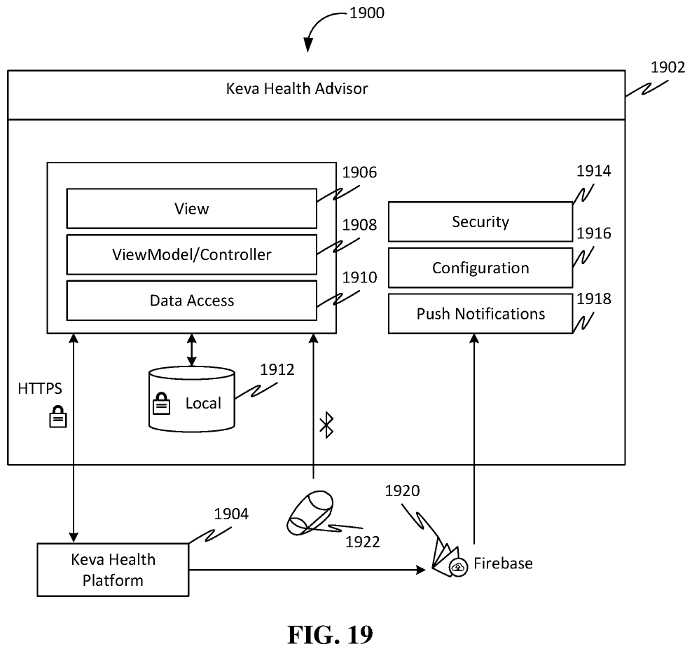

FIG. 19 illustrates an architecture 1900 of a Keva Health Advisor (also known as KevaTalk) application for facilitating the monitoring and managing of the respiratory diseases of the users, in accordance with some embodiments. Further, the architecture 1900 may include a Keva health advisor 1902, that communicates with a Keva health platform 1904. Further, the Keva health platform 1904 communicates with Firebase 1920 and a local data store 1912. Further, the Keva health advisor 1902 communicates with remote monitoring devices 1922. Further, the architecture 1900 may include a view 1906, a view model/controller 1908, and data access modules 1910. Further, the Keva Health Advisor application may handle security 1914, configuration 1916, and push notification 1918. Further, the push notification 1918 communicates with the Firebase 1920.

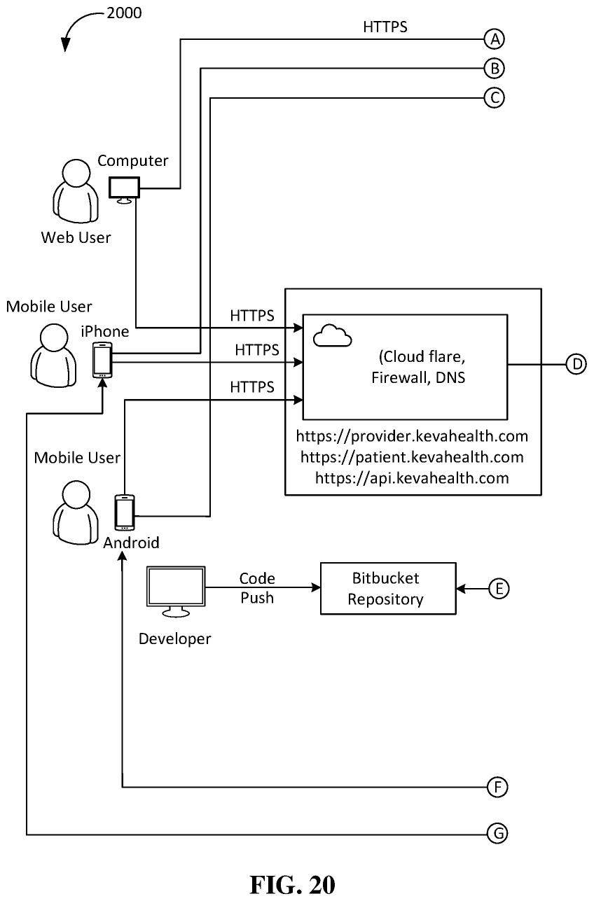

FIG. 20 illustrates a cloud-based system 2000 for facilitating monitoring and managing respiratory diseases of users, in accordance with some embodiments.

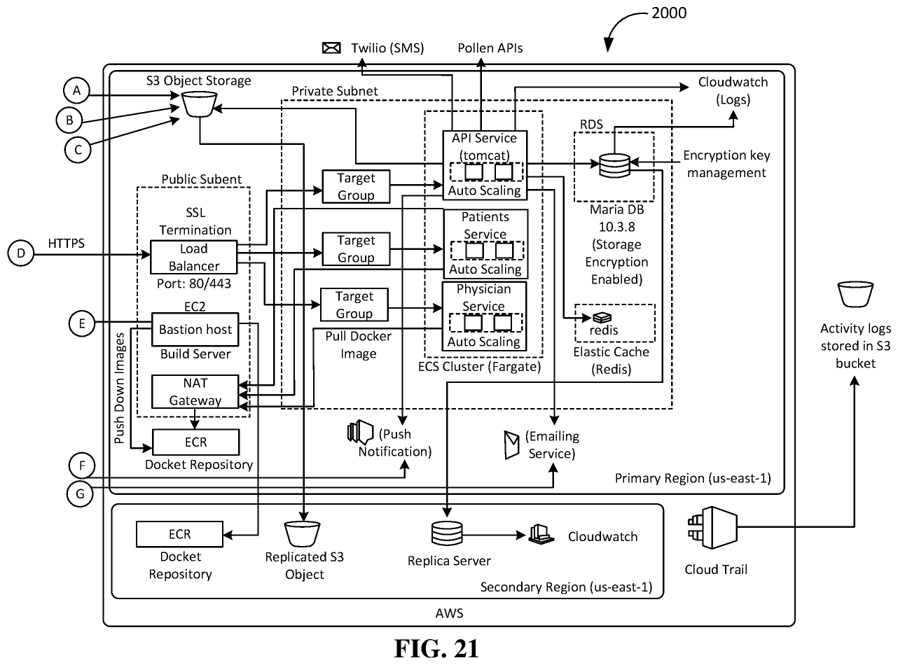

FIG. 21 illustrates the cloud-based system 2000 for facilitating the monitoring and managing of the respiratory diseases of the users, in accordance with some embodiments.

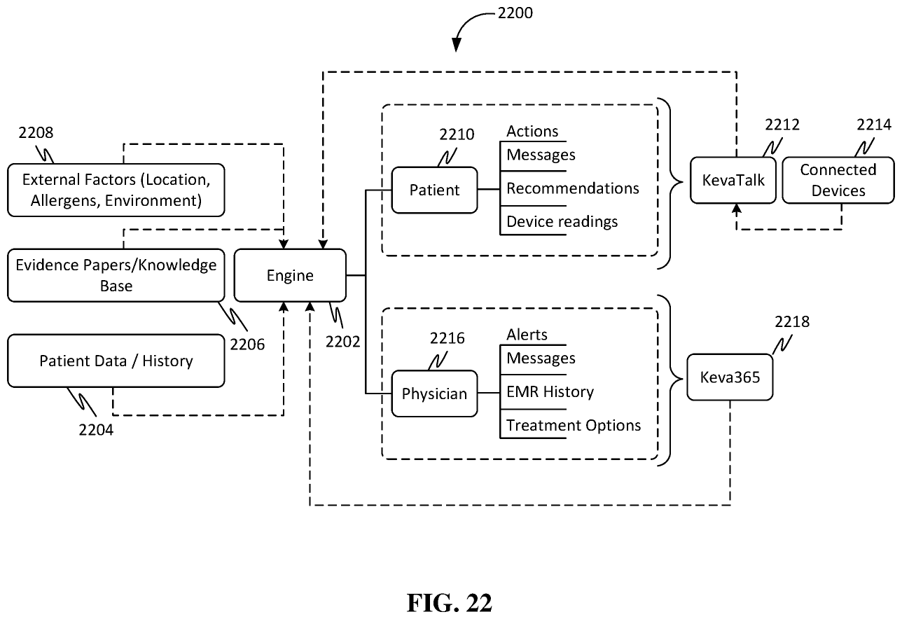

FIG. 22 illustrates a flow diagram of a method 2200 for facilitating monitoring and managing respiratory diseases of users, in accordance with some embodiments. Further, the method 2200 may include an engine 2202 receiving patient data/history 2204, evidence paper/knowledge base 2206, and external factors 2208 (such as environment, location, and allergen). The engine 2202 receives patient data 2210 via a Talk module (KevaTalk) 2212. The talk module 2212 receives data from connected devices 2214 and mobile devices, among others. The talk module 2212 generates actions, messages, recommendations, and stores device reading data. The physician 2216 receives alerts, messages, EMR history, and treatment options through a 365 module (Keva365) 2218 in communication with the engine 2202.

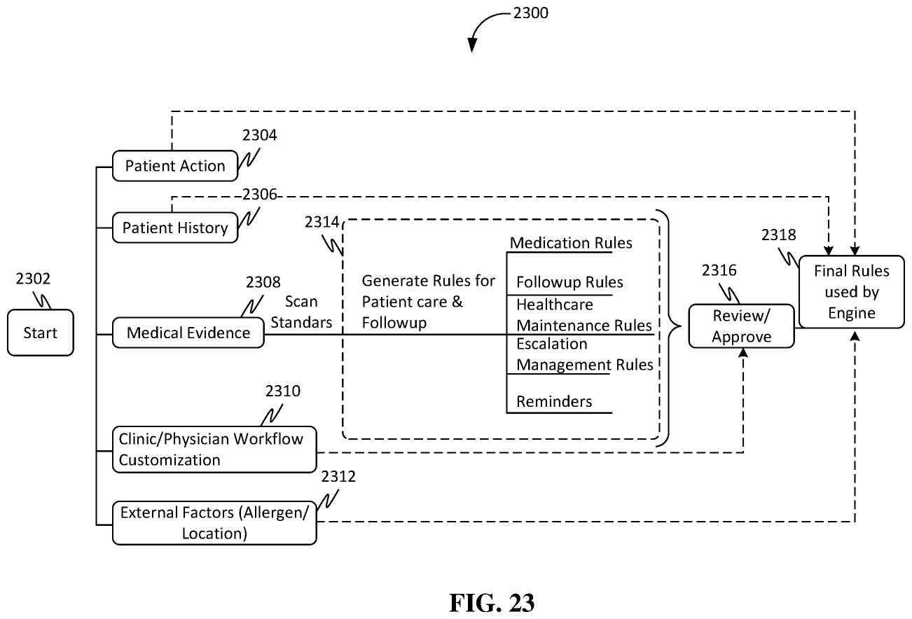

FIG. 23 illustrates a process 2300 for generating rules for the engine 2202 for facilitating the monitoring and managing of the respiratory diseases of the users, in accordance with some embodiments. Further, at 2302 the process 2300 starts. Further, at 2304 the process 2300 acquiring patient action. Further, at 2306 the process 2300 acquiring patient history. Further, at 2308 the process 2300 acquiring medical evidence. Further, at 2310 the process 2300 acquiring clinic/physician workflow. Further, at 2312 the process 2300 acquiring external factors such as allergens and location data. Further, at 2314 the process 2300 generates rules for patient care and follow up by deriving medication rules, follow rules, healthcare maintenance rules, escalation management rules, and reminders. Further, at 2316 the process 2300 reviews/approves. Further, at 2318 the process 2300 includes using the rules by the engine 2202.

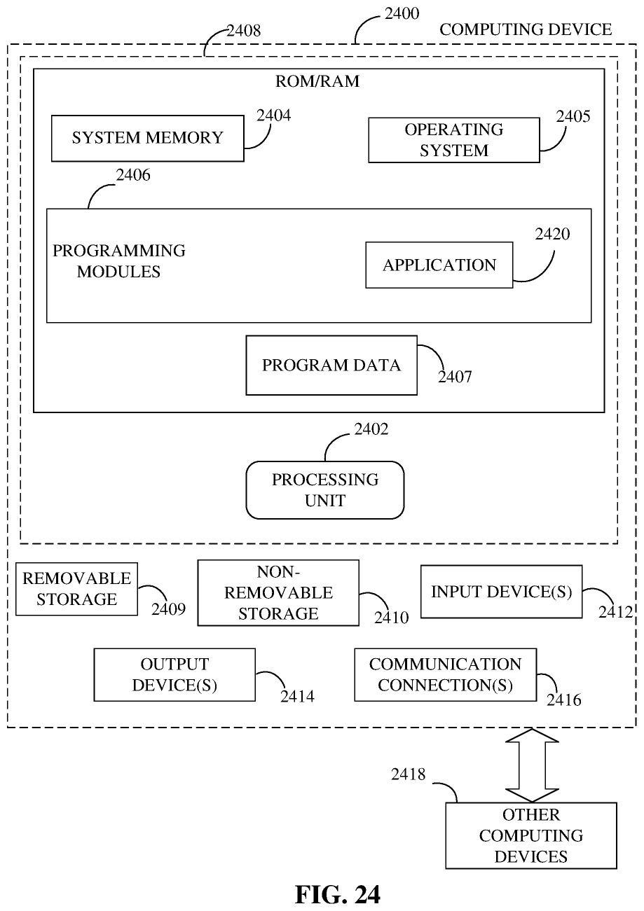

With reference to FIG. 24, a system consistent with an embodiment of the disclosure may include a computing device or cloud service, such as computing device 2400. In a basic configuration, computing device 2400 may include at least one processing unit 2402 and a system memory 2404. Depending on the configuration and type of computing device, system memory 2404 may comprise, but is not limited to, volatile (e.g. random-access memory (RAM)), non-volatile (e.g. read-only memory (ROM)), flash memory, or any combination. System memory 2404 may include operating system 2405, one or more programming modules 2406, and may include a program data 2407. Operating system 2405, for example, may be suitable for controlling computing device 2400's operation. In one embodiment, programming modules 2406 may include image-processing module, machine learning module. Furthermore, embodiments of the disclosure may be practiced in conjunction with a graphics library, other operating systems, or any other application program and is not limited to any particular application or system. This basic configuration is illustrated in FIG. 24 by those components within a dashed line 2408.

Computing device 2400 may have additional features or functionality. For example, computing device 2400 may also include additional data storage devices (removable and/or non-removable) such as, for example, magnetic disks, optical disks, or tape. Such additional storage is illustrated in FIG. 24 by a removable storage 2409 and a non-removable storage 2410. Computer storage media may include volatile and non-volatile, removable and non-removable media implemented in any method or technology for storage of information, such as computer-readable instructions, data structures, program modules, or other data. System memory 2404, removable storage 2409, and non-removable storage 2410 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 2400. Any such computer storage media may be part of device 2400. Computing device 2400 may also have input device(s) 2412 such as a keyboard, a mouse, a pen, a sound input device, a touch input device, a location sensor, a camera, a biometric sensor, etc. Output device(s) 2414 such as a display, speakers, a printer, etc. may also be included. The aforementioned devices are examples and others may be used.

Computing device 2400 may also contain a communication connection 2416 that may allow device 2400 to communicate with other computing devices 2418, such as over a network in a distributed computing environment, for example, an intranet or the Internet. Communication connection 2416 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 2404, including operating system 2405. While executing on processing unit 2402, programming modules 2406 (e.g., application 2420 such as a media player) may perform processes including, for example, one or more stages of methods, algorithms, systems, applications, servers, databases as described above. The aforementioned process is an example, and processing unit 2402 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

According to some aspects, a method for monitoring a respiratory disease is disclosed. Further, the method may include connecting to a respiratory device and receiving respiratory data for a patient from the device. Further, the method may include receiving a list of medications and an action plan. Further, the method may include tracking medication usage and adherence. Further, the method may include tracking objective measures including peak flow, FEV1, FEV6, FVC, and SpO2. Further, the method may include sharing a remote patient monitoring report with a healthcare professional. Further, the method may include providing a personalized chatbot and two-way messaging between the patient and healthcare professional. Further, the method may include tracking symptoms, and triggers, and reporting medication usage.

Further, according to some aspects, the method may include providing a real-time allergen report.

Further, according to some aspects, the method may include providing an educational portal where patients learn about health issues with resources in the portal.

Although the present disclosure has been explained in relation to its preferred embodiment, it is to be understood that many other possible modifications and variations can be made without departing from the spirit and scope of the disclosure.

What is claimed is:

1. A method for facilitating monitoring and managing diseases of users, the method comprising:
receiving, using a communication device, at least one disease data associated with at least one disease of at least one user from at least one device, wherein the at least one disease data comprises at least one of an action plan and a prescription for managing the at least one disease;
receiving, using the communication device, at least one user data associated with the at least one user from the at least one device, wherein the at least one user data comprises at least one health record of the at least one user;
analyzing, using a processing device, the at least one disease data and the at least one user data;
generating, using the processing device, a report of the at least one user based on the analyzing of the at least one disease data and the at least one user data;
processing, using the processing device, the report using at least one executable rule corresponding to the at least one disease;
determining, using the processing device, a status of the at least one user based on the processing of the report;
generating, using the processing device, a message for at least one of the at least one user and at least one healthcare provider of the at least one user based on the status, wherein the message comprises a guidance for the managing of the at least one disease;
transmitting, using the communication device, the message to at least one of at least one user device associated with the at least one user and at least one healthcare provider device associated with the at least one healthcare provider; and
storing, using a storage device, the message for at least one of the at least one user and the at least one healthcare provider.

2. The method of claim 1, wherein the at least one device further comprises at least one respiratory device, wherein the at least one respiratory device is configured for generating at least one respiratory data of the at least one user based on detecting at least one respiration characteristic of a respiration of the at least one user, wherein the at least one user data further comprises the at least one respiratory data.

3. The method of claim 1, wherein the at least one device further comprises at least one sensor, wherein the at least one sensor is configured for generating at least one user measurement of the at least one user based on detecting at least one of a physical state, a physiological state, and a biological state of the at least one user, wherein the at least one user data further comprises the at least one user measurement.

4. The method of claim 1, wherein the at least one device further comprises at least one external sensor, wherein the at least one external sensor is configured for generating at least one external sensor data associated with the at least one user based on detecting at least one external factor associated with the at least one user, wherein the at least one user data further comprises the at least one external sensor data.

5. The method of claim 1, wherein the at least one device further comprises at least one input device, wherein the at least one input device is configured for receiving at least one assessment of the at least one user from the at least one user, wherein the at least one user data further comprises the at least one assessment of the at least one user.

6. The method of claim 1, wherein the at least one disease data comprises at least one identifier of the at least one disease, wherein the method further comprises:
retrieving, using the storage device, at least one data associated with the managing of the at least one disease based on the at least one identifier; and
analyzing, using the processing device, the at least one data, wherein the generating of the report is further based on the analyzing of the at least one data.

7. The method of claim 1 further comprising:
transmitting, using the communication device, the report to the at least one healthcare provider device; and
receiving, using the communication device, at least one remark of the at least one healthcare provider corresponding to the report from the at least one healthcare provider device, wherein the processing of the report comprises processing the report and the at least one remark using the at least one executable rule, wherein the determining of the status is further based on the processing of the report and the at least one remark.

8. The method of claim 1 further comprising:
transmitting, using the communication device, the report to the at least one healthcare provider device;
receiving, using the communication device, at least one remark of the at least one healthcare provider corresponding to the report from the at least one healthcare provider device;
initiating, using the processing device, an interaction session between the at least one user and the at least one healthcare provider based on the at least one remark; and
extracting, using the processing device, at least one interaction between the at least one user and the at least one healthcare provider during the interaction session based on the initiating, wherein the processing of the report comprises processing the report and the at least one interaction using the at least one executable rule, wherein the determining of the status is further based on the processing of the report and the at least one interaction.

9. The method of claim 1, wherein the at least one disease data comprises at least one identifier of the at least one disease, wherein the method further comprising:
identifying, using the processing device, at least one guideline of at least one medical authority for the managing of the at least one disease based on the at least one identifier;
analyzing, using the processing device, the at least one guideline; and
generating, using the processing device, the at least one executable rule corresponding to the at least one disease based on the analyzing of the at least one guideline, wherein the processing of the report using the at least one executable rule is further based on the generating of the at least one executable rule.

10. A method for facilitating monitoring and managing diseases of users, the method comprising:
receiving, using a communication device, at least one disease data associated with at least one disease of at least one user from at least one device, wherein the at least one disease data comprises at least one of an action plan and a prescription for managing the at least one disease;
receiving, using the communication device, at least one user data associated with the at least one user from the at least one device, wherein the at least one user data comprises at least one health record of the at least one user, wherein the at least one device further comprises at least one respiratory device, wherein the at least one respiratory device is configured for generating at least one respiratory data of the at least one user based on detecting at least one respiration characteristic of a respiration of the at least one user, wherein the at least one user data further comprises the at least one respiratory data;
analyzing, using a processing device, the at least one disease data and the at least one user data;
generating, using the processing device, a report of the at least one user based on the analyzing of the at least one disease data and the at least one user data;
processing, using the processing device, the report using at least one executable rule corresponding to the at least one disease;
determining, using the processing device, a status of the at least one user based on the processing of the report;
generating, using the processing device, a message for at least one of the at least one user and at least one healthcare provider of the at least one user based on the status, wherein the message comprises a guidance for the managing of the at least one disease;
transmitting, using the communication device, the message to at least one of at least one user device associated with the at least one user and at least one healthcare provider device associated with the at least one healthcare provider; and
storing, using a storage device, the message for at least one of the at least one user and the at least one healthcare provider.

11. The method of claim 10 further comprising:
transmitting, using the communication device, the report to the at least one healthcare provider device; and
receiving, using the communication device, at least one remark of the at least one healthcare provider corresponding to the report from the at least one healthcare provider device, wherein the processing of the report comprises processing the report and the at least one remark using the at least one executable rule, wherein the determining of the status is further based on the processing of the report and the at least one remark.

12. A system for facilitating monitoring and managing diseases of users, the system comprising:
a communication device configured for:
receiving at least one disease data associated with at least one disease of at least one user from at least one device, wherein the at least one disease data comprises at least one of an action plan and a prescription for managing the at least one disease;
receiving at least one user data associated with the at least one user from the at least one device, wherein the at least one user data comprises at least one health record of the at least one user; and
transmitting a message to at least one of at least one user device associated with the at least one user and at least one healthcare provider device associated with at least one healthcare provider;
a processing device communicatively coupled with the communication device, wherein the processing device is configured for:
analyzing the at least one disease data and the at least one user data;
generating a report of the at least one user based on the analyzing of the at least one disease data and the at least one user data;
processing the report using at least one executable rule corresponding to the at least one disease;
determining a status of the at least one user based on the processing of the report; and
generating the message for at least one of the at least one user and the at least one healthcare provider of the at least one user based on the status, wherein the message comprises a guidance for the managing of the at least one disease; and
a storage device communicatively coupled with the processing device,
wherein the storage device is configured for storing the message for at least one of the at least one user and the at least one healthcare provider.

13. The system of claim 12, wherein the at least one device further comprises at least one respiratory device, wherein the at least one respiratory device is configured for generating at least one respiratory data of the at least one user based on detecting at least one respiration characteristic of a respiration of the at least one user, wherein the at least one user data further comprises the at least one respiratory data.

14. The system of claim 12, wherein the at least one device further comprises at least one sensor, wherein the at least one sensor is configured for generating at least one user measurement of the at least one user based on detecting at least one of a physical state, a physiological state, and a biological state of the at least one user, wherein the at least one user data further comprises the at least one user measurement.

15. The system of claim 12, wherein the at least one device further comprises at least one external sensor, wherein the at least one external sensor is configured for generating at least one external sensor data associated with the at least one user based on detecting at least one external factor associated with the at least one user, wherein the at least one user data further comprises the at least one external sensor data.

16. The system of claim 12, wherein the at least one device further comprises at least one input device, wherein the at least one input device is configured for receiving at least one assessment of the at least one user from the at least one user, wherein the at least one user data further comprises the at least one assessment of the at least one user.

17. The system of claim 12, wherein the at least one disease data comprises at least one identifier of the at least one disease, wherein the storage device is further configured for retrieving at least one data associated with the managing of the at least one disease based on the at least one identifier, wherein the processing device is further configured for analyzing the at least one data, wherein the generating of the report is further based on the analyzing of the at least one data.

18. The system of claim 12, wherein the communication device is further configured for:
transmitting the report to the at least one healthcare provider device; and
receiving at least one remark of the at least one healthcare provider corresponding to the report from the at least one healthcare provider device, wherein the processing of the report comprises processing the report and the at least one remark using the at least one executable rule, wherein the determining of the status is further based on the processing of the report and the at least one remark.

19. The system of claim 12, wherein the communication device is further configured for:
transmitting the report to the at least one healthcare provider device; and
receiving at least one remark of the at least one healthcare provider corresponding to the report from the at least one healthcare provider device, wherein the processing device is further configured for:
initiating an interaction session between the at least one user and the at least one healthcare provider based on the at least one remark; and
extracting at least one interaction between the at least one user and the at least one healthcare provider during the interaction session based on the initiating, wherein the processing of the report comprises processing the report and the at least one interaction using the at least one executable rule, wherein the determining of the status is further based on the processing of the report and the at least one interaction.

20. The system of claim 11, wherein the at least one disease data comprises at least one identifier of the at least one disease, wherein the processing device is further configured for:
identifying at least one guideline of at least one medical authority for the managing of the at least one disease based on the at least one identifier;
analyzing the at least one guideline; and
generating the at least one executable rule corresponding to the at least one disease based on the analyzing of the at least one guideline, wherein the processing of the report using the at least one executable rule is further based on the generating of the at least one executable rule.
